(19) United States
(12) Patent Application Publication
(10) Pub. No.: US 2026/0305965 A1
(43) Pub. Date: Oct. 8, 2026

(54) ELECTRONIC DEVICE AND DRIVING METHOD THEREOF

(71) Applicant: SAMSUNG DISPLAY CO., LTD., YONGIN-SI (KR)

(72) Inventor: Chul KIM, Yongin-si (KR)

(21) Appl. No.: 19/254,215

(22) Filed: Jun. 30, 2025

(30) Foreign Application Priority Data
Apr. 4, 2025 (KR) 10-2025-0044007

(51) Int. Cl. A61B 5/024; A61B 5/00; A61B 5/021; G06F 3/041; G06F 3/044; G06T 7/00; G09G 3/32; G09G 3/3233

(52) U.S. Cl. CPC A61B 5/02427; A61B 5/02108; A61B 5/7203; G06F 3/0416; G06T 7/0016; G09G 3/3233; G06F 3/0446; G06F 2203/04104; G06T 2207/30196; G09G 3/32; G09G 2300/0819; G09G 2300/0842; G09G 2300/0861; G09G 2354/00; G09G 2360/145; G09G 2380/08

(57) ABSTRACT

An electronic device includes a display panel with a plurality of sub-pixels and photo sensors, a display driving circuit to drive them, and a touch panel with touch electrodes driven by a touch driving circuit. A processor controls the display driving circuit over a plurality of frame periods to generate a left hand finger image in an area where the user's left hand is detected, generate a right hand finger image in an area where the user's right hand is detected, and compare the generated images. By analyzing the left and right hand images over time and space, the device can extract physiological features, such as systolic and diastolic peaks, and assess the user's cardiovascular condition. The electronic device may enable comparative bio-signal sensing between both sides of the body.

### CROSS-REFERENCE TO RELATED APPLICATION

This U.S. patent application claims priority under 35 U.S.C. § 119(a) to Korean Patent Application No. 10-2025-0044007 filed in the Korean Intellectual Property Office on Apr. 4, 2025, the disclosure of which is incorporated by reference in its entirety herein.

### 1. TECHNICAL FIELD

Embodiments of the present disclosure relate to an electronic device and a driving method thereof.

### 2. Description of Related Art

With advancements in information technology, display devices serving as interfaces between users and information, have become increasingly important. Accordingly, the adoption of various display devices such as liquid crystal display devices and organic light-emitting display devices, continues to grow.

For example, the electronic device may support a high-security biometric authentication function by utilizing the user's biometric information such as a fingerprint.

In recent years, various types of software have been developed for execution on electronic devices to assist a medical equipment in managing a user's health.

Electronic devices have been developed that continuously track health-related data related such as a user's bio-signals through advances in software and structural enhancements to display devices.

Health-related information, such as user's cardiovascular data, may be collected through the electronic device. Recently, various approaches have been proposed to enhance the accuracy of the collected information. However, many existing methods rely on analyzing bio-signals from a single point of contact or a single body part, often using static or instantaneous measurements that do not account for variations across time or between different sides of the body. These approaches fail to capture subtle asymmetries or dynamic physiological changes that are important for accurate cardiovascular assessment.

### SUMMARY OF THE INVENTION

At least one embodiment of the disclosure provides an electronic device and a driving method thereof that enable comparison of information acquired from a user's left and right body parts over a specific period of time in terms of time and space. For example, the comparison may be performed along a spatiotemporal axis, allowing the system to extract and evaluate physiological features from both sides of the body over time, thereby facilitating more comprehensive assessment of the user's cardiovascular condition.

An embodiment of the present disclosure provides an electronic display device including a display panel having a plurality of sub-pixels and a plurality of photo sensors; a display driving circuit configured to drive the plurality of sub-pixels and the plurality of photo sensors; a touch panel positioned to overlap the display panel and having a plurality of touch electrodes; a touch driving circuit to drive the plurality of touch electrodes to respectively detect an area where a finger of a user's left hand has been touched and an area where a finger of the user's right hand has been touched; and a processor controlling the display driving circuit over a plurality of frame periods to generate a left hand finger image of the user in an area where the finger of the user's left hand is detected, generate right hand finger image of the user in an area where the finger of the user's right hand is detected, and comparing the generated left and right hand finger images.

The processor may generate a plurality of images including the left hand finger image, and a plurality of images including the right hand finger image during the plurality of frame periods.

The processor may detect an area having a highest signal-to-noise ratio in the plurality of images including the left hand finger image, and may detect an area having a highest signal-to-noise ratio in a plurality of images including the right hand finger image.

The processor may calculate a first feature value around an area having a highest signal-to-noise ratio in the left hand finger image, and may calculate a second feature value around an area having a highest signal-to-noise ratio in the right hand finger image.

The first feature value may include information associated with a systolic peak and information associated with a diastolic peak which are measured from the user's left hand, and the second feature value may include information associated with a systolic peak and information associated with a diastolic peak which are measured from the user's right hand.

The processor may generate bio-signal sensing data based on the first feature value and the second feature value.

The bio-signal sensing data may include information associated with a cardiovascular system of the user.

The processor may include a touch area detector to receive a digital value from the display driving circuit, to generate left hand images including the left hand finger image of the user in an area where the finger of the user's left hand is detected, and to generate right hand images including the right hand finger image of the user in an area where the finger of the user's right hand is detected; a left hand space-time axis interpreter configured to generate a first feature value from the left hand images generated over a plurality of frame periods in spatial coordinates; a right hand space-time axis interpreter configured to generate a second feature value from the right hand images generated over a plurality of frame periods in spatial coordinates; and a two-hand comparator configured to generate bio-signal sensing data based on the first feature value and the second feature value.

The left hand space-time axis interpreter may detect an area having a highest signal-to-noise ratio in the left hand images, and the right hand space-time axis interpreter may detect an area having a highest signal-to-noise ratio in the right hand images.

The left hand space-time axis interpreter may generate the first feature value centered on a first center coordinate calculated to have the highest signal-to-noise ratio in the left hand images, and the right hand space-time axis interpreter may generate the first feature value centered on a second center coordinate calculated to have the highest signal-to-noise ratio in the right hand images.

The two-hand comparator may compare positions of the first center coordinate and the second center coordinate.

The two-hand comparator may compare the first feature value with the second feature value.

An embodiment of the present disclosure provides a driving method of an electronic device including a memory storing an application and a processor executing the application, and the driving method includes generating left hand images including a left hand finger image of a user in an area where a finger of the user's left hand is detected to have been touched; generating right hand images including a right hand finger image of the user in an area where a finger of the user's right hand is detected to have been touched; generating a first feature value from the left hand images generated over a plurality of frame periods in spatial coordinates; generating a second feature value from the right hand images generated over a plurality of frame periods in spatial coordinates; and generating bio-signal sensing data based on the first feature value and the second feature value.

In the generating a first feature value from the left hand images, the processor may detect an area having a highest signal-to-noise ratio in the left hand images, and, in the generating a second feature value from the right hand images, the processor may detect an area having a highest signal-to-noise ratio in the right hand images.

In the generating a first feature value from the left hand images, the processor may generate the first feature value centered on a first center coordinate calculated to have the highest signal-to-noise ratio in the left hand images, and, in the generating a second feature value from the right hand images, the processor may generate the second feature value centered on a second center coordinate calculated to have the highest signal-to-noise ratio in the right hand images.

In the generating bio-signal sensing data, the processor may compare positions of the first center coordinate and the second center coordinate.

In the generating bio-signal sensing data, the processor may compare the first feature value and the second feature value.

The first feature value may include information associated with a systolic peak and information associated with a diastolic peak which are measured from the user's left hand, and the second feature value may include information associated with a systolic peak and information associated with a diastolic peak which are measured from the user's right hand.

The bio-signal sensing data may include information associated with a cardiovascular system of the user.

Each of the left hand finger image and the right hand finger image may be generated based on a difference in reflected light incident on the electronic device according to contraction and relaxation of blood vessels of the user.

### BRIEF DESCRIPTION OF THE DRAWINGS

FIG. 1 is a schematic system diagram of an electronic device according to embodiments of the present disclosure.

FIG. 2 is a block diagram illustrating driving of an electronic device according to embodiments of the present disclosure.

FIG. 3A is an exemplary cross-sectional view of the display device of FIG. 2.

FIG. 3B is another exemplary cross-sectional view of the display device of FIG. 2.

FIG. 4 is a system block diagram of a display device according to embodiments of the present disclosure.

FIG. 5 is a diagram illustrating a touch panel and a touch driving circuit of FIG. 2.

FIG. 6 is an example of the touch panel of FIG. 5.

FIG. 7 is an enlarged view of a first area of FIG. 6.

FIG. 8 is a cross-sectional view taken along line I-I′ of FIG. 7.

FIG. 9 is an enlarged view of a second area of FIG. 6.

FIG. 10 is a cross-sectional view taken along line II-II′ of FIG. 9.

FIG. 11 is a layout diagram illustrating a display area according to an embodiment.

FIG. 12 is an equivalent circuit diagram of a sub-pixel and a photo sensor according to an embodiment.

FIG. 13 is an equivalent circuit diagram of a sub-pixel and a photo sensor according to another embodiment.

FIG. 14 is a diagram illustrating some of configurations of a sub-pixel and a photo sensor in a cross-sectional view of a display device according to embodiments of the present disclosure.

FIG. 15 is an equivalent circuit diagram of a readout circuit according to embodiments of the present disclosure.

FIG. 16 is a timing diagram of a display device for sensing a photo sensor at a first sensing resolution in case that a first mode is selected in embodiments of the present disclosure.

FIGS. 17 and 18 are timing diagrams of a display device for sensing a photo sensor at a second sensing resolution in case that a second mode is selected in embodiments of the present disclosure.

FIG. 19 is a diagram illustrating a screen displayed in a bio-signal sensing mode and a process of detecting a plurality of body part touch areas.

FIG. 20 is a graph illustrating one pulse signal detected in real in a biometric information measurement period and a process of detecting an inaccurate pulse signal.

FIG. 21 is a diagram illustrating a detected contact area of a finger in contact with a display area during a biometric information measurement period.

FIG. 22 is an example of images generated over a plurality of frame periods.

FIG. 23 is an example of a plurality of images generated over a plurality of frame periods.

FIG. 24 is a block diagram of a processor according to embodiments of the present disclosure.

FIG. 25 is a block diagram of an electronic device according to an embodiment of the present disclosure.

FIG. 26 is a schematic diagram of an electronic device according to various embodiments.

### DETAILED DESCRIPTION

Hereinafter, various embodiments of the present disclosure will be described in detail with reference to the accompanying drawings so that those skilled in the art can easily implement the embodiments. The present disclosure may be implemented in various different forms and is not limited to the embodiments described herein.

In order to clearly explain the present disclosure, parts unrelated to the description are omitted, and the same or similar components are denoted by the same reference numerals throughout the specification. Therefore, the reference numerals described above can also be used in other drawings.

Also, the expression “same” in the description may mean “substantially the same”. That is, it may be the same to the extent that those skilled in the art can understand that they are the same. Other expressions may also be expressions in which “substantially” is omitted.

The terms first, second, etc. may be used to describe various components, but the components should not be limited by the terms. The terms are used only for the purpose of distinguishing one component from another. For example, a first component may be termed a second component, and similarly, a second component may also be termed a first component, without departing from the scope of the present disclosure. A singular expression includes plural expressions unless the context clearly dictates otherwise.

Terms such as “below”, “down”, “above”, “on”, etc. are used to describe the association of the configurations shown in the figures. The terms are relative concepts and will be described with reference to the directions shown in the drawings.

It is to be understood that the terms “comprise” or “have” and the like are intended to designate the presence of a feature, number, step, operation, component, part, or combination thereof described in the specification, and do not preclude the presence or possibility of addition of one or more other features or numbers, steps, operations, components, parts, or combinations thereof.

At least one embodiment of the disclosure relates to an electronic device and driving method that enhances biometric sensing by performing spatiotemporal analysis of touch-based images from both the left and right hands of a user. Specifically, when the user touches the display with both hands, a processor controls the display driving circuit over multiple frame periods to generate a left hand finger image and a right hand finger image in their respective detected touch areas. These images are analyzed across time and space to extract feature values, such as systolic and diastolic peaks, from regions having the highest signal-to-noise ratios. By comparing these features between the two hands, the system generates bio-signal sensing data that provides a more complete and diverse representation of the user's cardiovascular condition. This dual-hand, temporal, and spatial comparison distinguishes the invention from conventional systems that rely on single-point or single-frame biometric analysis.

Hereinafter, embodiments of the present disclosure will be described in detail with reference to the accompanying drawings.

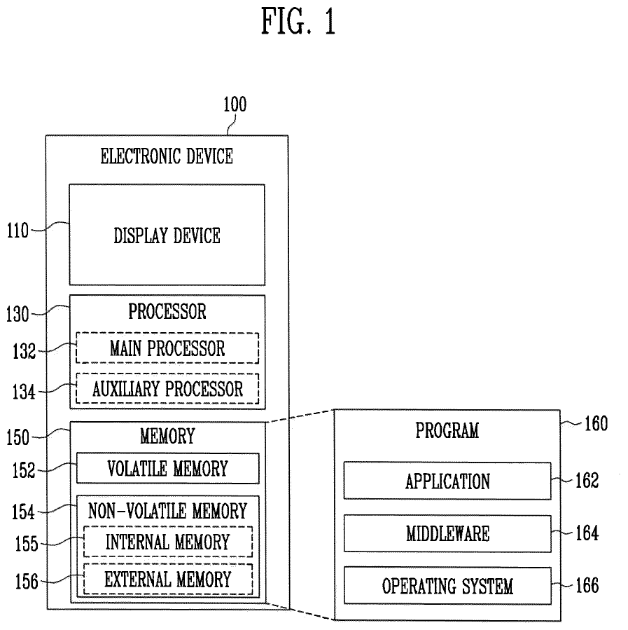

FIG. 1 is a schematic system diagram of an electronic device 100 according to embodiments of the present disclosure.

Referring to FIG. 1, an electronic device 100 according to embodiments of the present disclosure may include a display device 110, a processor 130 and a memory 150.

The display device 110 may visually provide information to the outside (e.g., a user) of the electronic device 100. The display device 110 may include, for example, a display panel and a driving circuit. The display device 110 according to embodiments of the present disclosure may include a touch sensor set to detect a touch and/or a pressure sensor set to measure intensity of a force generated by the touch.

The processor 130 may, for example, execute software (e.g., a program 160) to control at least one other component (e.g., a hardware component or a software component) of the electronic device 100 coupled to the processor 130, and may perform various data processing or operations. According to embodiments of the present disclosure, as at least part of data processing or operation, the processor 130 may store data received from another component (e.g., the display device 110) in a volatile memory 152, process instructions or data stored in the volatile memory 152, and store result data in a non-volatile memory 154. According to embodiments of the present disclosure, the processor 130 may include a main processor 132 (e.g., a central processing unit or an application processor) or an auxiliary processor 134 (e.g., a graphics processing unit (GPU), a neural processing unit (NPU), an image signal processor, a sensor hub processor, a communication processor, etc.) operable independently of or in conjunction with the main processor 132. For example, in case that the electronic device 100 includes a main processor 132 and an auxiliary processor 134, the auxiliary processor 134 may use lower power than the main processor 132 or may be set to be specialized for a specified function. The auxiliary processor 134 may be implemented separately from, or as part of, the main processor 132.

The auxiliary processor 134 may control at least a portion of a function or states associated with at least one component (e.g., the display device 110) of the components of the electronic device 100, for example, on behalf of the main processor 132 while the main processor 132 is in an inactive (e.g., sleep) state, or in conjunction with the main processor 132 when the main processor 132 was in an active state (e.g., executing an application). According to embodiments of the present disclosure, the auxiliary processor 134 (e.g., an image signal processor or a communication processor) may be implemented as part of another functionally relevant component (e.g., a camera module (not shown), a communication module (not shown), etc.). According to embodiments of the present disclosure, the auxiliary processor 134 (e.g., a neural network processing device) may include a hardware structure specialized in processing of an artificial intelligence model. The artificial intelligence models may be created through machine learning.

The memory 150 may store various data used by at least one component (e.g., the processor 130) of the electronic device 100. Data may include, for example, software (e.g., a program 160) and input data or output data for instructions associated with the software. The memory 150 may include a volatile memory 152 or a non-volatile memory 154. The non-volatile memory 154 may include an embedded memory 155. The non-volatile memory 154 may further include an external memory 156.

The program 160 may be stored as software in the memory 150 and may include, for example, an application 162, a middleware 164 and an operating system 166.

The electronic device 100 according to embodiments of the present disclosure may be referred to as a mobile station, a mobile equipment (ME), a user equipment (UE), a user terminal (UT), a subscriber station (SS), a wireless device, a handheld device or an access terminal (AT). The electronic device 100 according to embodiments of the present disclosure may be, for example, a device having a communication function, such as a mobile phone, a personal digital assistant (PDA), a smart phone, a wireless MODEM, or a laptop computer.

The electronic device 100 according to embodiments of the present disclosure may include a power management module (not shown) configured to manage power supplied to the electronic device 100. The power management module may be implemented, for example, as at least a part of a power management integrated circuit (PMIC).

At least some of the components of the electronic device 100 according to embodiments of the present disclosure may be connected to each other through a communication method between peripheral devices (e.g., a bus, a general purpose input and output (GPIO), a serial peripheral interface (SPI) or a mobile industry processor interface (MIPI)), and may exchange signals (e.g., commands or data) with each other.

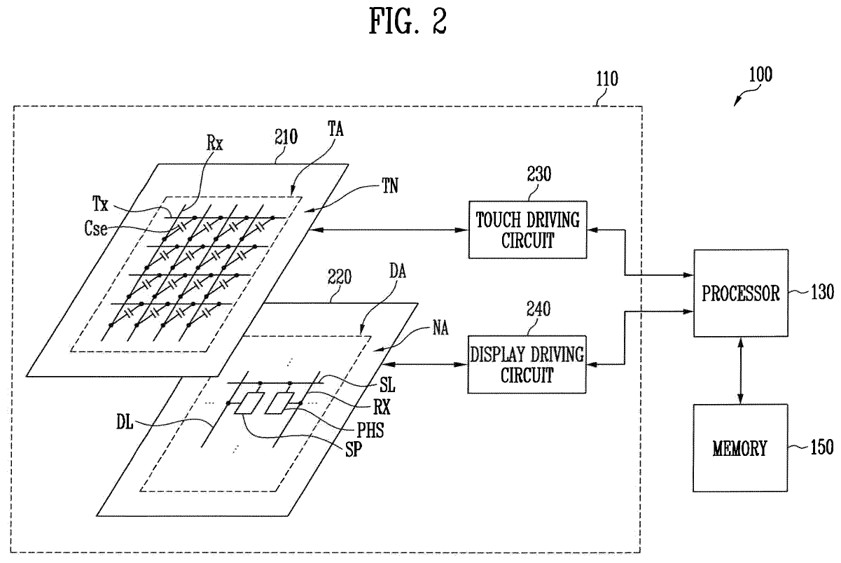

FIG. 2 is a block diagram illustrating driving of an electronic device 100 according to embodiments of the present disclosure.

Referring to FIG. 2, an electronic device 100 according to embodiments of the present disclosure may include a display device 110, a processor 130, and a memory 150.

Referring to FIG. 2, a display device 110 according to embodiments of the present disclosure may include a touch panel 210, a display panel 220, a touch driving circuit 230 and a display driving circuit 240.

The display device 110 according to embodiments of the present disclosure may be applied to a mobile communication terminal. The display device 110 may constitute a portable terminal by being disposed on a bracket and/or a case together with electronic modules, a camera module and a power module mounted on a main board. However, the display device 110 is not limited thereto. For example, the display device 110 may be applied to an electronic device 100 such as a television, a notebook, a monitor, a billboard or an Internet on Things (IoT) device, as well as an electronic device 100 (e.g., a small or medium-sized electronic device) such as a mobile phone, a smart phone, a tablet personal computer (PC), navigation, a smart watch, a watch phone, a Ultra-Mobile Personal Computer (UMPC), a Portable Multimedia Player (PMP), an electronic notebook or an electronic book.

Referring to FIG. 2, a touch panel 210 and a display panel 220 are shown separately from each other. however, this is merely for functionally distinguishing the touch panel 210 and the display panel 220 in the display device 110. In an embodiment, the touch panel 210 according to embodiments of the present disclosure may be formed in a separate process from the display panel 220 so that the touch panel 210 and the display panel 220 are coupled to each other (e.g., the touch panel 220 is attached and coupled to one surface of the display panel 220). In the above embodiment, the touch panel 210 may be formed in an add-on type. In another embodiment, the touch panel 210 according to embodiments of the present disclosure may be formed in one process (e.g., a process of manufacturing the display panel 220) with the display panel 220. In the above embodiment, the touch panel 210 may be formed in an in-cell type.

The touch panel 210 may be provided on one surface of the display panel 220. For example, the touch panel 210 may be disposed on one surface (e.g., an upper surface) of both surfaces of the display panel 220 in a direction in which an image is emitted. In another embodiment, the touch panel 210 may be formed directly on at least one of both surfaces of the display panel 220 or may be formed inside the display panel 220. For example, the touch panel 210 may be formed directly on an outer surface of an upper substrate or a lower substrate of the display panel 220 (i.e., an upper surface of the upper substrate or a lower surface of the lower substrate), or may be formed directly onto an inner surface of the upper substrate (i.e., a lower surface of the upper substrate) or an inner surface of the lower substrate (i.e., an upper surface of the lower substrate).

The touch panel 210 may include a touch active area TA capable of sensing a touch input, and a touch inactive area TN located in a peripheral area of the touch active area TA (e.g., an edge area of the touch active area TA). According to an embodiment, the touch active area TA may be located to correspond to a display area DA of the display panel 220.

According to an embodiment, the touch panel 210 may be located to overlap at least one area with the display panel 220. For example, the touch active area TA of the touch panel 210 may be located on the display area DA of the display panel 220. According to an embodiment, at least one electrode for detecting a touch input may be disposed in the touch active area TA. The at least one electrode for detecting a touch input may include, for example, a first touch electrode Tx and a second touch electrode Rx. The first touch electrode Tx and the second touch electrode Rx may be provided on the display area DA of the display panel 220.

Wirings for electrically connecting the at least one electrode provided in the touch active area TA to the touch driving circuit 230 may be disposed in the touch inactive area TN. For example, wirings for electrically connecting the first touch electrode Tx and the second touch electrode Rx to the touch driving circuit 230 may be disposed in the touch inactive area TN. The touch inactive area TN may be located to correspond to a non-display area NA of the display panel 220.

The touch panel 210 may include at least one first touch electrode Tx and a second touch electrode Rx provided in the touch active area TA. For example, the touch panel 210 may include the first touch electrode Tx and the second touch electrode Rx that intersects the first touch electrode TX. In an embodiment, the first touch electrode Tx may extend along a first direction. In an embodiment, the second touch electrode Rx may extend along a second direction crossing the first direction while being insulated from the first touch electrode Tx by an insulating film. A capacitance Cse may be formed between the first touch electrode Tx and the second touch electrode Rx. The capacitance Cse may be changed when a touch input occurs at or around a corresponding point. By detecting a change in the capacitance Cse, a touch input and/or a position (or coordinate) at which the touch input occurred may be detected.

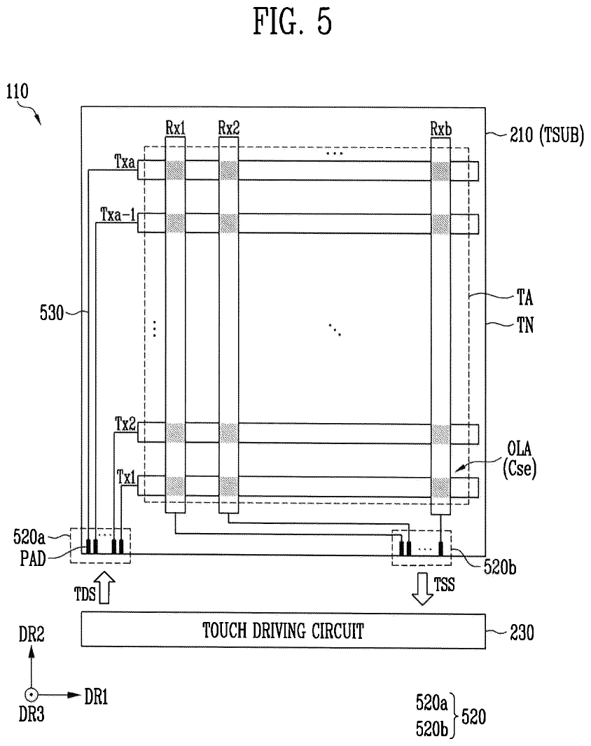

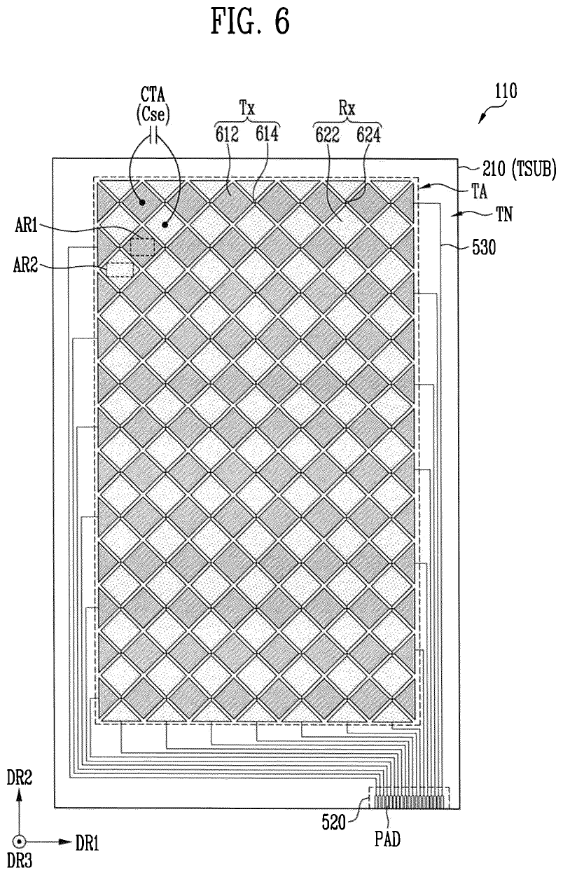

A shape, size, and/or arrangement direction of the first touch electrode Tx and the second touch electrode Rx are not particularly limited. Exemplary embodiments related thereto are described with reference to FIGS. 5 and 6.

The display panel 220 may include a display area DA and a non-display area NA located in a peripheral area of the display area DA (e.g., an edge area of the display area DA).

A signal line SL and a data line DL may be located in the display area DA. A sub-pixel SP may be located in the display area DA, and the sub-pixel SP is electrically connected to the signal line SL and the data line DL. Various driving signals for driving the sub-pixel SP and/or wirings for supplying power may be provided in the non-display area NA.

In the embodiments of the present disclosure, the type of the display panel 220 is not particularly limited. For example, in embodiments of the present disclosure, the display panel 220 may be a self-luminous display panel. In the above embodiment, the display panel 220 may include a plurality of light-emitting elements. For example, a light-emitting element may be selected as an organic light-emitting diode. For example, the light-emitting element may be selected as an inorganic light-emitting diode, such as a micro LED (light-emitting diode), a quantum dot light-emitting diode. For example, the light-emitting element may be an element in which an organic material and an inorganic material are combined.

For example, in embodiments of the present disclosure, the display panel 220 may be a non-light-emitting display panel such as a liquid crystal display panel (LCD panel), an electro-permanent display panel (EPD panel), or an electro-wetting display panel (EWD panel). In case that the display panel 220 is the non-light-emitting display panel, the display device 110 may further include a backlight unit for supplying light to the display panel 220.

The touch driving circuit 230 may be connected (e.g., electrically connected) to touch panel 210, and transmit a signal input to the touch panel 210 or receive a signal output from touch panel 210. The touch driving circuit 230 may supply a touch driving signal to the touch panel 210, thereafter, receive a touch sensing signal (or a touch detection signal) corresponding to the touch driving signal from the touch panel 210 to detect a touch input. According to an embodiment, the touch driving circuit 230 may be integrated in one integrated circuit (IC), but may be divided and disposed in two or more integrated circuits (ICs) according to functions. According to an embodiment, the touch driving circuit 230 may simultaneously (or sequentially) supply a touch driving signal to the plurality of first touch electrodes Tx. The touch driving circuit 230 may simultaneously (or sequentially) receive a touch sensing signal from the plurality of second touch electrodes Rx. The touch driving circuit 230 may receive the touch sensing signal from the touch panel 210 and detect whether a touch is input and/or a touch coordinate by performing signal processing on the touch sensing signal.

The display driving circuit 240 may be connected to (e.g., electrically connected to) the display panel 220, and supply a signal input to the display panel 220 or receive a signal output from the display panel 220. The display driving circuit 240 may supply a scan signal or a light emission signal to the signal line SL, and may supply a data signal (or a data voltage) to the data line DL.

The processor 130 may provide a signal to the display device 110 or receive a signal generated by the display device 110. The processor 130 may provide a signal to the display device 110 with reference to the memory 150. The processor 130 may store data in the memory 150 based on a signal received from the display device 110. For example, information for fingerprint authentication of a user of the electronic device 100 (e.g., a reference fingerprint image for fingerprint authentication) may be stored in the memory 150. For example, the memory 150 may store bio-signal sensing data (e.g., blood pressure, heart rate, oxygen saturation, etc.) of a user of the electronic device 100. A code for executing an application may be stored in the memory 150, and the processor 130 may execute the application with reference to the memory 150 to provide a signal to the display device 110.

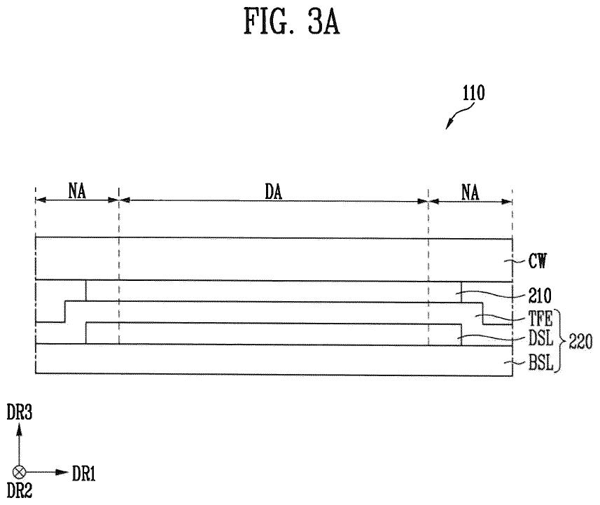

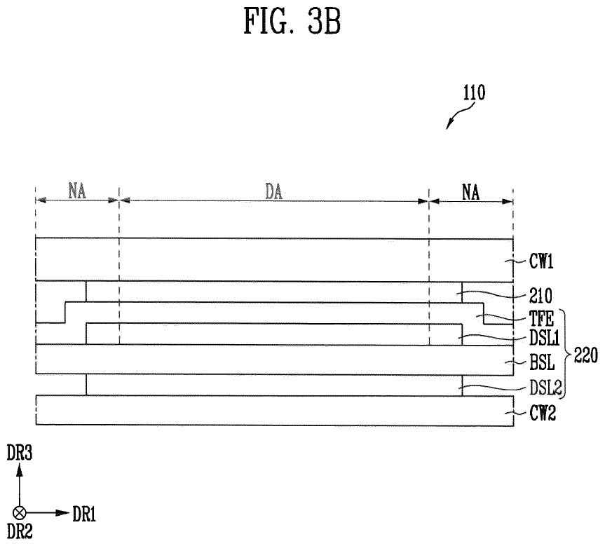

FIG. 3A is an exemplary cross-sectional view of the display device 110 of FIG. 2.

Referring to FIG. 3A, the display device 110 may include a display panel 220 and a touch panel 210 disposed on the display panel 220. A cover window CW may be disposed on the touch panel 210.

In FIG. 3A, an embodiment in which the display panel 220 is a self-luminous display panel including a light-emitting element is described as an example. However, embodiments of the present disclosure are not limited thereto.

The display panel 220 may include a base substrate BSL, an element layer DSL and an encapsulation layer TFE.

The base substrate BSL may support the element layer DSL. The base substrate BSL may provide a plane defined by a first direction DR1 and a second direction DR2, and support the element layer DSL on the plane. The base substrate BSL may include an insulating material. As an example of the insulating material, at least one among glass, quartz, ceramic, and plastic may be included. In an embodiment, the base substrate BSL may be a rigid substrate. In another embodiment, the base substrate BSL may be a flexible substrate.

The element layer DSL may be positioned on the base substrate BSL (e.g., in a third direction DR3 from the base substrate BSL). The element layer DSL may include a sub-pixel SP (see FIG. 2) and a photo sensor PHS (see FIG. 2) located on the base substrate BSL. The sub-pixel may include a light-emitting element and a pixel circuit for driving the light-emitting element. The photo sensor may include a light-receiving element and a sensor circuit for driving the light-receiving element. Each of the pixel circuit and the sensor circuit may include a switching element (e.g., a transistor) and a storage element (e.g., a capacitor). A transistor, a capacitor, a light-emitting element, and a light-receiving element may be disposed in the element layer DSL. A signal line for providing a signal to the sub-pixel and the photo sensor and a power line for providing a power voltage may be disposed in the element layer DSL. The signal line may include a scan line configured to transmit a scan signal to the sub-pixel and the photo sensor. A data line configured to supply a data voltage to the sub-pixel may be disposed in the element layer DSL. The sub-pixel and the photo sensor may be disposed in the display area DA. A common electrode (e.g., a cathode electrode of a light-emitting element and a light-receiving element) may be formed in the element layer DSL.

The encapsulation layer TFE may be disposed on the element layer DSL. The encapsulation layer TFE may protect the element layer DSL from external moisture and/or oxygen. The encapsulation layer TFE may include two or more thin film layers formed on the element layer DSL. For example, the encapsulation layer TFE may include an inorganic thin film layer formed on the element layer DSL, an organic thin film layer formed on the inorganic thin film layer, and an inorganic thin film layer disposed on the organic thin film layer. According to an embodiment, the encapsulation layer TFE may be formed of a glass substrate to cover the element layer DSL. The encapsulation layer TFE may cover the element layer DSL in the display area DA and the non-display area NA.

The touch panel 210 may be disposed on the encapsulation layer TFE. In an embodiment, the touch panel 210 may be formed directly on the encapsulation layer TFE. In another embodiment, the touch panel 210 may be formed through a separate process from the display panel 220 and disposed (e.g., attached) on the encapsulation layer TFE. The touch panel 210 may include a touch active area TA, and at least a portion of the touch active area TA may overlap the display area DA. The touch panel 210 may include a touch inactive area TN (see FIG. 2), and at least a portion of the touch inactive area TN may overlap the non-display area NA.

The cover window CW may be disposed on the touch panel 210. The cover window CW may protect the display panel 220 and the touch panel 210 from an external impact. The cover window CW may be embodied as a film of a light-transmitting (e.g., transparent) material, for example glass and/or plastic.

According to an embodiment, the display device 110 may further include one or more optical layers (e.g., an anti-glare layer, a polarizing plate, a color filter and a liquid crystal).

FIG. 3B is another exemplary cross-sectional view of the display device 110 of FIG. 2.

The display panel 220 may include a base substrate BSL, a first element layer DSL1, a second element layer DSL2 and an encapsulation layer TFE.

The base substrate BSL may support the first element layer DSL1 and the second element layer DSL2. The base substrate BSL may provide a plane defined by the first direction DR1 and the second direction DR2, and support the first element layer DSL1 and the second element layer DSL2 on the plane.

The first element layer DSL1 may be positioned on the base substrate BSL (e.g., in a third direction DR3 from the base substrate BSL). The first element layer DSL1 may include a sub-pixel SP (see FIG. 2) and a photo sensor PHS (see FIG. 2) positioned on the base substrate BSL. The sub-pixel may include a light-emitting element and a pixel circuit for driving the light-emitting element. The photo sensor may include a light-receiving element and a sensor circuit for driving the light-receiving element. Each of the pixel circuit and the sensor circuit may include a switching element (e.g., a transistor) and a storage element (e.g., a capacitor). A transistor, a capacitor, a light-emitting element, and a light-receiving element may be disposed in the first element layer DSL1. A signal line for providing a signal to the sub-pixel and the photo sensor and a power line for providing a power voltage may be disposed in the first element layer DSL1. The signal line may include a scan line configured to transmit a scan signal to the sub-pixel and the photo sensor. The first element layer DSL1 may include a data line configured to provide a data signal to a sub-pixel. The sub-pixel and the photo sensor may be disposed in the display area DA. A common electrode (e.g., a cathode electrode of a light-emitting element and a light-receiving element) may be formed in the first element layer DSL1.

The second element layer DSL2 may be located below the base substrate BSL (e.g., in a direction opposite to the third direction DR3 from the base substrate BSL). The second element layer DSL2 may include a light-emitting element and a light-receiving element positioned under the base substrate BSL. A signal line through which a signal for controlling driving timings of the light-emitting element and the light-receiving element is transmitted may be disposed in the second element layer DSL2. A power line for providing a power voltage to the light-emitting element and the light-receiving element may be disposed in the second element layer DSL2. The signal line may include a scan line for controlling a light emission timing of the light-emitting element and controlling a sensing timing of the light-receiving element. The light-emitting element and the light-receiving element may be disposed in the display area DA. A common electrode (e.g., a cathode electrode of the light-emitting element and the light-receiving element) may be formed in the second element layer DSL2. In an embodiment, an encapsulation layer covering the second element layer DSL2 may be further provided. However, embodiments of the present disclosure are not limited thereto.

The touch panel 210 may be disposed on the encapsulation layer TFE. In an embodiment, the touch panel 210 may be formed directly on the encapsulation layer TFE. In another embodiment, the touch panel 210 may be formed through a separate process from the display panel 220 and disposed (e.g., attached) on the encapsulation layer TFE. The touch panel 210 may include a touch active area TA, and at least a portion of the touch active area TA may overlap the display area DA. The touch panel 210 may include a touch inactive area TN (see FIG. 2), and at least a portion of the touch inactive area TN may overlap the non-display area NA.

The first cover window CW1 may be disposed on the touch panel 210. The first cover window CW1 may protect the display panel 220 and the touch panel 210 from an external impact. The first cover window CW1 may be implemented as a film of a light-transmitting (e.g. transparent) material, for example glass and/or plastic.

The second cover window CW2 may be disposed under the second element layer DSL2. The second cover window CW2 may protect the second element layer DSL2 from an external impact. The second cover window CW2 may be implemented as a film of a light-transmitting (e.g., transparent) material, for example, glass and/or plastic.

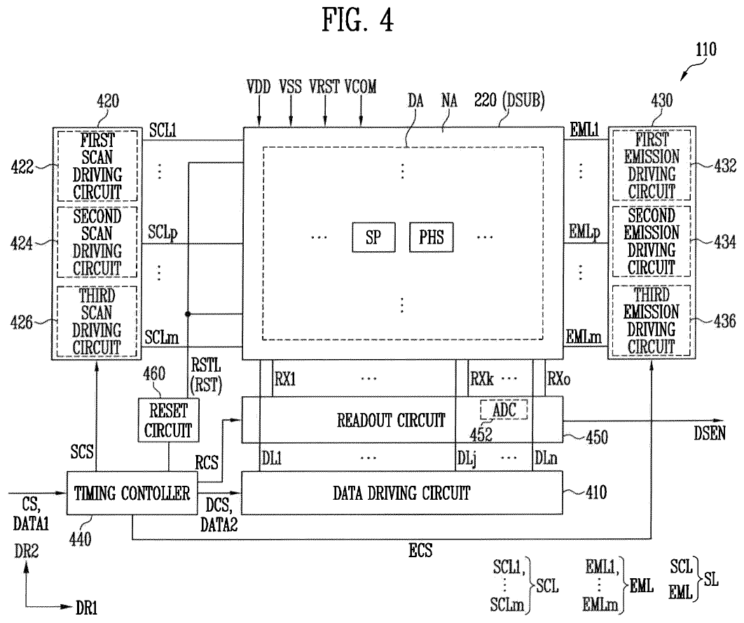

FIG. 4 is a system block diagram of a display device 110 according to embodiments of the present disclosure.

Referring to FIG. 4, a display device 110 according to embodiments of the present disclosure may include a display panel 220, a data driving circuit 410, a scan driving circuit 420, an emission driving circuit 430, a timing controller 440, a readout circuit 450 and a reset circuit 460.

The above-described display driving circuit 240 (see FIG. 2) may include the data driving circuit 410, the scan driving circuit 420, the emission driving circuit 430, the timing controller 440, the readout circuit 450 and the reset circuit 460.

One or more sub-pixels SP may be disposed on the display panel 220. One or more photo sensors PHS may be disposed on the display panel 220. One or more signal lines configured to provide signals to the sub-pixel SP and the photo sensor PHS may be disposed on the display panel 220. One or more data lines configured to provide a data voltage to the sub-pixel SP may be disposed on the display panel 220. A power line configured to supply a power voltage to the sub-pixel SP and the photo sensor PHS may be disposed on the display panel 220. The sub-pixel SP and the photo sensor PHS may be disposed on a display substrate DSUB. The signal line, the data lines DL1 to DLn, and the power line may be disposed on the display substrate DSUB. The display substrate DSUB may include the base substrate BSL described above (see FIGS. 3A and 3B).

The power voltage may include, for example, a first power voltage VDD, a second power voltage VSS, a third power voltage VRST and a fourth power voltage VCOM. The power voltage is commonly input to the plurality of sub-pixels SP and/or the plurality of photo sensors PHS, and is also referred to as a common voltage. The power voltage may be generated, for example, in a power management integrated circuit.

A plurality of data lines DL1 to DLn (n is an integer of 2 or more) may be arranged in the display panel 220. The plurality of data lines DL1 to DLn may be arranged to extend in a second direction DR2 in the display panel 220. The second direction DR2 may be, for example, a direction (e.g., a column direction) connecting an upper side and a lower side of the display panel 220. The second direction DR2 may be, for example, a direction (e.g., a row direction) connecting a left side and a right side of the display panel 220. Hereinafter, for convenience of description, the second direction DR2 is described as a direction connecting the upper side and the lower side of the display panel 220, for example, but embodiments of the present disclosure are not limited thereto.

A plurality of scan lines SCL1 to SCLm (m is an integer of 2 or more) may be arranged in the display panel 220. The plurality of scan lines SCL1 to SCLm may be arranged to extend in a first direction DR1 in the display panel 220. The first direction DR1 may be, for example, a direction connecting a left side and a right side of the display panel 220. The first direction DR1 may be, for example, a direction connecting an upper side and a lower side of the display panel 220. The first direction DR1 may be, for example, a direction intersecting the second direction DR2 (e.g., perpendicular to the second direction DR2). Hereinafter, for convenience of description, the first direction DR1 is described as a direction connecting the left side and the right side of the display panel 220, for example, but embodiments of the present disclosure are not limited thereto.

On the other hand, being arranged to extend in the second direction DR2 may mean being arranged to extend in a direction connecting the upper side and the lower side as a whole, and partially extending in a direction different from the second direction DR2, is not excluded. For example, in embodiments of the present disclosure, at least one data line of the plurality of data lines DL1 to DLn may be designed to extend partially bypassing in a direction different from the second direction DR2 in order to avoid a specific area (e.g., an area in which transmittance is set high). A meaning of being arranged to extend in the first direction DR1 may also be understood in the same sense as the meaning of being arranged to extend in the second direction DR2.

A plurality of light emission control lines EML1 to EMLm may be arranged in the display panel 220. The plurality of light emission control lines EML1 to EMLm may be arranged to extend in the first direction DR1 in the display panel 220.

A plurality of sensing lines RX1 to RXo (o is an integer of 2 or more) may be arranged in the display panel 220. A plurality of sensing lines RX1 to RXo may be arranged to extend in the second direction DR2 in the display panel 220.

One or more reset control lines RSTL may be arranged in the display panel 220.

The sub-pixel SP may be electrically connected to any one of the plurality of data lines DL1 to DLn. The sub-pixel SP may be electrically connected to at least one scan line among the plurality of scan lines SCL1 to SCLm. The sub-pixel SP may be electrically connected to at least one light emission control line among the plurality of light emission control lines EML1 to EMLm.

The photo sensor PHS may be electrically connected to any one of the plurality of sensing lines RX1 to RXo. According to an embodiment, the photo sensor PHS may be electrically connected to the reset control line RSTL. According to an embodiment, the photo sensor PHS may be electrically connected to at least one scan line among the plurality of scan lines SCL1 to SCLm. According to an embodiment, the photo sensor PHS may be electrically connected to at least one light emission control line among the plurality of light emission control lines EML1 to EMLm.

The sub-pixel SP and the photo sensor PHS may be electrically connected to any one of the plurality of scan lines SCL1 to SCLm.

According to an embodiment, a plurality of sub-pixels SP may be arranged in a matrix type in the display panel 220. For example, the plurality of sub-pixels SP may be arranged in an RGB type, or may be arranged in a rhombus-shaped PENTILE™ structure.

The data driving circuit 410 may be configured to supply a data voltage to the plurality of data lines DL1 to DLn. The data driving circuit 410 may receive second image data DATA2 and supply a data voltage corresponding to the second image data TATA2 to the plurality of data lines DL1 to DLn in response to a data driving circuit control signal DCS.

The scan driving circuit 420 may be configured to supply a scan signal to the plurality of scan lines SCL1 to SCLm. According to an embodiment, the scan driving circuit 420 may be configured to sequentially supply scan signals to the plurality of scan lines SCL1 to SCLm, but is not limited thereto. For example, the scan driving circuit 420 may include a plurality of sub-scan driving circuits 422, 424, and 426. The sub-scan driving circuits 422, 424, and 426 may be configured to spatially divide the display area DA to provide a scan signal. For example, the first scan driving circuit 422 may supply a scan signal to an upper ⅓ area (e.g., an area including a first scan line SCL1) of the display area DA. The second scan driving circuit 424 may supply a scan signal to a central ⅓ area (e.g., an area including a p-th scan line SCLp) of the display area DA. The third scan driving circuit 426 may supply a scan signal to a lower ⅓ area (e.g., an area including a m-th scan line SCLm) of the display area DA. The scan driving circuit 420 may receive the scan driving circuit control signal SCS and supply the scan signal to the plurality of scan lines SCL1 to SCLm According to Timing.

The emission driving circuit 430 may be configured to supply a light emission control signal to the plurality of light emission control lines EML1 to EMLm. According to an embodiment, the emission driving circuit 430 may be configured to sequentially supply light emission control signals to the plurality of light emission control lines EML1 to EMLm, but is not limited thereto. For example, the emission driving circuit 430 may include a plurality of sub-emission driving circuits 432, 434, and 436. The sub-emission driving circuits 432, 434, and 436 may be configured to spatially divide the display area DA to provide a light emission control signal. For example, the first emission driving circuit 432 may supply a light emission control signal to an upper ⅓ area of the display area DA (e.g., an area including a first light emission control line EML1). The second emission driving circuit 434 may supply a light emission control signal to a central ⅓ area (e.g., an area including a p-th light emission control line EMLp) of the display area DA. The third emission driving circuit 436 may supply a light emission control signal to a lower ⅓ area (e.g., an area including a m-th light emission control line EMLm) of the display area DA. The emission driving circuit 430 may receive an emission driving circuit control signal ECS and supply the light-emitting control signal to the plurality of light emission control lines EML1 to EMLm according to timing.

The timing controller 440 may receive a control signal CS and first image data DATA1 from the outside (e.g., the processor 130 (see FIG. 1)). The timing controller 440 may generate the data driving circuit control signal DCS, the scan driving circuit control signal SCS, the emission driving circuit control signal ECS, the second image data DATA2 and a readout circuit control signal RCS based on the control signal CS and the first image data DATA1 which are input.

The readout circuit 450 may be electrically connected to the plurality of sensing lines RX1 to RXo. The readout circuit 450 may be configured to sense the plurality of photo sensors PHS through the plurality of sensing lines RX1 to RXo. For example, according to the design of the readout circuit 450, the readout circuit 450 may integrate a current flowing through at least one of the plurality of sensing lines RX1 to RXo or sense a voltage of at least one of the plurality of the sensing lines RX1 to RXo. The readout circuit 450 may further include a multiplexer configured to integrate a current (or sense a voltage) of at least one of the plurality of sensing lines RX1 to RXo. Hereinafter, for convenience of description, the readout circuit 450 will be described as an example in which a current sensing method is applied, but embodiments of the present disclosure are not limited thereto.

The readout circuit 450 may include an analog-to-digital converter (ADC) 452 configured to convert an analog voltage to a digital value DSEN.

The reset circuit 460 may be configured to supply a reset signal RST to the plurality of photo sensors PHS. When the reset signal RST is supplied to the photo sensor PHS, an electrical connection between the photo sensors PHS and the sensing lines RX1 to RXo may be broken. A timing at which the reset circuit 460 outputs the reset signal RST may be controlled by the timing controller 440.

One or more circuits constituting the display driving circuit 240 (see FIG. 2) may be disposed in the display device 110 in the form of an integrated circuit. For example, the data driving circuit 410 and the readout circuit 450 may be disposed in the display device 110 in the form of a Source Readout Integrated Circuit (SRIC). One or more circuits constituting the display driving circuit 240 may be formed together in the process of forming the display panel 220. For example, the scan driver circuit 420 and/or the emission driving circuit 430 may be formed together in a process of forming one or more circuit elements (e.g., transistors, etc.) included in the sub-pixel SP and the photo sensor PHS.

The data driving circuit 410, the scan driving circuit 420, the emission driving circuit 430, and the timing controller 440 are merely classified according to functions, and two or more components may be functionally distinguished within one integrated circuit. For example, the data driving circuit 410 and the timing controller 440 may be implemented as one integrated circuit, but may be functionally separated within the integrated circuit. For example, the scan driving circuit 420 and the emission driving circuit 430 may be implemented as one integrated circuit, but may be functionally separated within the integrated circuit.

FIG. 5 is a diagram illustrating the touch panel 210 and the touch driving circuit 230 of FIG. 2.

The display device 110 may include the touch panel 210 and the touch driving circuit 230.

Referring to FIG. 5, the touch panel 210 according to embodiments of the present disclosure may include a touch substrate TSUB, first touch electrodes Tx1, Tx2 to Txa-1, Txa (hereinafter, Tx1 to Txa, where a is an integer of 2 or more), second touch electrodes Rx1, Rx2 to Rxb (hereinafter, Rx1 to Rxb, where b is an integer of 2 or more), a pad portion 520, and a touch wiring 530.

The touch substrate TSBU may be a substrate serving as a substrate of the touch panel 210, and may be a rigid or flexible substrate. For example, the touch substrate TSUB may be a rigid substrate including glass or tempered glass. For example, the touch substrate TSUB may be a flexible substrate including a thin film made of a flexible plastic material. The first touch electrodes Tx1 to Txa, the second touch electrodes Rx1 to Rxb, the pad portion 520 and the touch wiring 530

### May Be Disposed on the Touch Substrate Tsub. According to an Embodiment, the

touch substrate TUSB may be omitted or replaced with another configuration. For example, in case that the first touch electrodes Tx1 to Txa and the second touch electrodes Rx1 to Rxb are directly formed on the display panel 220 (see FIG. 2), the touch substrate TSUB may be replaced with the base substrate BSL (see FIGS. 3A and 3B) or the encapsulation layer TFE (see FIG. 3A and FIG. 3B) described above.

The first touch electrodes Tx1 to Txa may extend along a first direction (e.g., a direction transverse from left to right of the touch panel 210, or a row direction). Referring to FIG. 5, each of the plurality of first touch electrodes Tx1 to Txa may be arranged to extend in a row direction in the touch panel 210.

The second touch electrodes Rx1 to Rxb may extend along a second direction (e.g., a direction transverse from an upper side to a lower side of the touch panel 210, or a column direction). Referring to FIG. 5, each of the plurality of second touch electrodes Rx1 to Rxb may be arranged to extend in a column direction in the touch panel 210.

The touch panel 210 according to embodiments of the present disclosure may be driven in a mutual capacitance manner. In the mutual capacitance manner, any one of the first touch electrodes Tx1 to Txa and the second touch electrodes Rx1 to Rxb may be a touch driving electrode. In the mutual capacitance manner, the other of the first touch electrodes Tx1 to Txa and the second touch electrodes Rx1 to Rxb may be a touch sensing electrode. A touch driving signal TDS may be input to the touch driving electrode. A touch sensing signal TSS may be output through the touch sensing electrode. In an embodiment, the first touch electrodes Tx1 to Txa may receive the touch driving signal TDS for touch driving, and the second touch electrodes Rx1 to Rxb may output the touch sensing signal TSS generated in response to the touch driving signal TDS. In another embodiment, the second touch electrodes Rx1 to Rxb may receive the touch driving signal TDS, and the first touch electrodes Tx1 to Txa may output the touch sensing signal TSS generated in response to the touch driving signal TDS. Hereinafter, for convenience of description, an embodiment in which the touch driving signal TDS is input to the first touch electrodes Tx1 to Txa and the touch sensing signal TSS is output through the second touch electrodes Rx1 to Rxb will be described as an example. However, embodiments of the present disclosure are not limited thereto.

A capacitance Cse may be formed between the first touch electrodes Tx1 to Txa and the second touch electrodes Rx1 to Rxb. For example, referring to FIG. 5, the first touch electrodes Tx1 to Txa and the second touch electrodes Rx1 to Rxb may overlap each other in a vertical direction (e.g., a third direction DR3) in an overlapping area OLA. In the overlapping area OLA, the first touch electrodes Tx1 to Txa may be one electrode of a capacitor, and the second touch electrodes Rx1 to Rxb may be the other electrode of the capacitor. The capacitance Cse may be formed between the first touch electrodes Tx1 to Txa and the second touch electrodes Rx1 to Rxb in the overlapping area OLA. When an object (e.g., a finger of a human or a stylus pen) approaches the touch panel 210, the capacitance Cse value may be changed due to the object. The touch driving circuit 230 may detect whether a touch is input and/or touch coordinates based on a value of the changed capacitance Cse.

The pad portion 520 may include one or more pads PAD. The pads PAD may connect (e.g., electrically connect) the first touch electrodes Tx1 to Txa and the second touch electrodes Rx1 to Rxb with the touch driving circuit 230. For example, the touch driving signal TDS may be input to the first touch electrodes Tx1 to Txa through a first pad portion 520a. For example, the touch sensing signal TSS may be output from the second touch electrodes Rx1 to Rxb through a second pad portion 520b.

The touch wiring 530 may connect (e.g., electrically connect) the first pad portion 520a and the first touch electrodes Tx1 to Txa. Alternatively, the touch wiring 530 may connect (e.g., electrically connect) between the second pad portion 520b and the second touch electrodes Rx1 to Rxb.

In an embodiment, the pad portion 520 and the touch wiring 530 may be located in the touch inactive area TN.

FIG. 6 is an example of the touch panel 210 of FIG. 5.

A first touch electrode Tx of the touch panel 210 according to embodiments of the present disclosure may include a first sensing cell 612 and a first connection electrode 614. A second touch electrode Rx may include a second sensing cell 622 and a second connection electrode 624.

The first touch electrode Tx may extend along the first direction DR1. The first touch electrode Tx may include a plurality of first sensing cells 612 and a first connection electrode 614 configured to connect the plurality of first sensing cells 612 to each other. In an embodiment, the first connection electrode 614 may be integrally configured with the first sensing cell 612. In an embodiment, the first connection electrode 614 may be configured with a connection pattern in the form of a bridge.

The second touch electrode Rx may extend along the second direction DR2. The second touch electrode Rx may include a plurality of second sensing cells 622 and a second connection electrode 624 configured to connect the plurality of second sensing cells 622 to each other. In an embodiment, the second connection electrode 624 may be integrally configured with the second sensing cell 622. In an embodiment, the second connection electrode 624 may be configured in a bridge-shaped connection pattern.

The first connection electrode 614 and the second connection electrode 624 may be positioned to overlap each other in the vertical direction (e.g., the third direction DR3). The first connection electrode 614 and the second connection electrode 624 may be insulated from each other by an insulating layer.

Referring to FIG. 6, a capacitance Cse may be formed between the first touch electrode Tx and the second touch electrode Rx. For example, the first touch electrode Tx may be one electrode of a capacitor, and the second touch electrode Rx adjacent to the first touch electrode Tx may be the other electrode of the capacitor. A capacitance Cse may be formed in an adjacent area CTA between the first touch electrode Tx and the second touch electrode Rx. An air (e.g., atmosphere) between the first touch electrode Tx and the second touch electrode Rx may be an insulating material of the capacitor. Thereby, whether a touch is input and/or touch coordinates may be detected in a mutual capacitance manner.

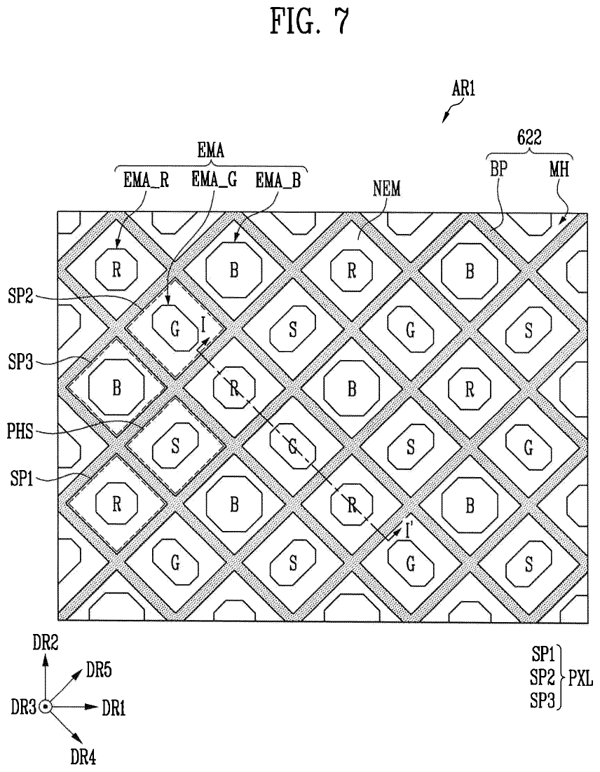

FIG. 7 is an enlarged view of a first area AR1 of FIG. 6.

The pixel PXL may include a plurality of sub-pixels SP1, SP2, and SP3. Each of the plurality of sub-pixels SP1, SP2, and SP3 may include an emission area and a non-emission area NEM. Referring to FIG. 7, the pixel PXL may include a first sub-pixel SP1, a second sub-pixel SP2, and a third sub-pixel SP3.

The emission area is an area in which light may be emitted. The light-receiving area is an area capable of receiving light. In an embodiment in which the sub-pixels SP1, SP2, and SP3 include a light-emitting element, the emission area may correspond to an area where the light-emitting element is located. A first emission area EMA_R may be located in the first sub-pixel SP1. A second emission area EMA_G may be located in the second sub-pixel SP2. A third emission area EMA_B may be located in the third sub-pixel Sp3.

The non-emissive area NEM may be located in a periphery (e.g., edge area) of the emission areas EMA_R, EMA_G, and EMA_B and a periphery of the light-receiving area (e.g., edge area). The sub-pixels SP1, SP2, and SP3 may be distinguished from each other by the non-emission area NEM. In an embodiment, a pixel driving circuit of each of the first to third sub-pixels SP1 to SP3 may be located in at least some of areas overlapping the non-emission area NEM. In an embodiment, a sensor circuit of the photo sensor PHS may be located in at least some of areas overlapping the non-emission area NEM. In an embodiment, the non-emission area NEM may correspond to an area in which a pixel defining layer or a black matrix is located. The emission areas EMA_R, EMA_G, and EMA_B, the light-receiving area, and the non-emission area NEM may constitute the above-Described display area DA (see FIG. 2).

The first sub-pixel SP1 may be configured to emit light in a first wavelength band (e.g., a red wavelength band). The second sub-pixel SP2 may emit light in a second wavelength band (e.g., a green wavelength band). The third sub-pixel SP3 may emit light in a third wavelength band (e.g., a blue wavelength band). The red wavelength band may be a wavelength band of approximately 600 nm (nanometers) to 750 nm. The green wavelength band may be a wavelength band of approximately 480 nm to 560 nm. The blue wavelength band may be a wavelength band of approximately 370 nm to 460 nm. In an embodiment, the first sub-pixel SP1 may be a red sub-pixel that emits light in a red wavelength band. The second sub-pixel SP2 may be a green sub-pixel that emits light in a green wavelength band. The third sub-pixel SP3 may be a blue sub-pixel that emits light in a blue wavelength band. However, embodiments of the present disclosure are not limited thereto.

In the pixel PXL, a plurality of sub-pixels SP1, SP2, and SP3 may be arranged in various ways. For example, referring to FIG. 7, the first sub-pixel SP1 and the third sub-pixel SP3 may be alternately arranged along the first direction DR1 in any one of a plurality of pixel rows. Referring to FIG. 7, the second sub-pixel SP2 and the photo sensor PHS may be alternately arranged in another row among the plurality of pixel rows. Referring to FIG. 7, the first sub-pixel SP1 and the second sub-pixel SP2 may be alternately arranged along a fourth direction DR4 in a diagonal direction intersecting each of the first direction DR1 and the second direction DR2. Referring to FIG. 7, the third sub-pixel SP3 and the photo sensor PHS may be alternately arranged along the fourth direction DR4. A row in which the first sub-pixel SP1 and the second sub-pixel SP2 are alternately arranged and a row in which the third sub-pixel SP3 and the photo sensor PHS are alternately arranged may be located adjacent to each other in a fifth direction DR5. The fifth direction DR5 may intersect the fourth direction DR4. For example, the fifth direction DR5 may be a direction perpendicular to the fourth direction DR4.

An area of the emission area of any one of the first sub-pixel SP1, the second sub-pixel SP2, and the third sub-pixel SP3 may be different from those of the other two. For example, an area of the emission area EMA_B of the third sub-pixel SP3 may be larger than an area of the emission area EMA_R of the first sub-pixel SP1. For example, an area of the emission area EMA_R of the first sub-pixel SP1 may be larger than an area of the emission area EMA_G of the second sub-pixel SP2. However, embodiments of the present disclosure are not limited thereto. For example, the areas of the emission areas of the first sub-pixel SP1, the second sub-pixel SP2, and the third sub-pixel SP3 may all be designed to be the same.

Shapes of the emission area and the light-receiving area may be variously designed. For example, referring to FIG. 7, the shapes of the emission areas EMA_R, EMA_G, and EMA_B and the light-receiving area of the photo sensor PHS are shown to be octagonal, but may have a shape such as a circle, another polygon, or a polygon with rounded corners.

In an embodiment, the shapes of the first emission area EMA_R and the third emission area EMA_B may be regular octagons. However, embodiments of the present disclosure are not limited thereto.

In an embodiment, the shape of the second emission area EMA_G may be an octagon long in the fourth direction DR4. However, embodiments of the present disclosure are not limited thereto.

In an embodiment, the shape of the photo sensor PHS (e.g., the light-receiving area of the photo sensor PHS) may be an octagon long in the fifth direction DR5. However, embodiments of the present disclosure are not limited thereto.

Referring to FIG. 7, the first touch electrode Tx may include a mesh hole MH and a body portion BP.

The mesh hole MH may overlap the emission areas EMA_R, EMA_G, and EMA_B and the light-receiving area of the photo sensor PHS in a thickness direction (e.g., the third direction DR3). An area of the mesh hole MH may be larger than an area of the emission areas EMA_R, EMA_G, and EMA_B and the light-receiving area of the photo sensor PHS.

The body portion BP may overlap the non-emission area NEM in the thickness direction (e.g., the third direction DR3). The width of the body portion BP may be smaller than the width of the non-emission area NEM.

Through the above structure, light output from the emission areas EMA_R, EMA_G, and EMA_B may effectively pass through the first touch electrode Tx (see FIG. 6). Through the above structure, light incident on the light-receiving area of the photo sensor PHS may effectively transmit through the first touch electrode Tx.

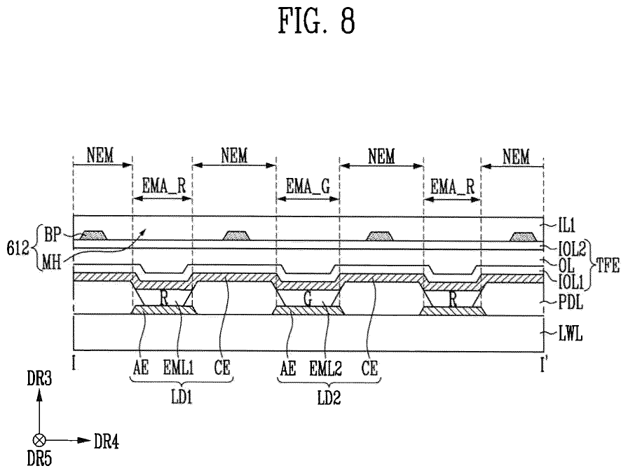

FIG. 8 is a cross-sectional view taken along line I-I′ of FIG. 7.

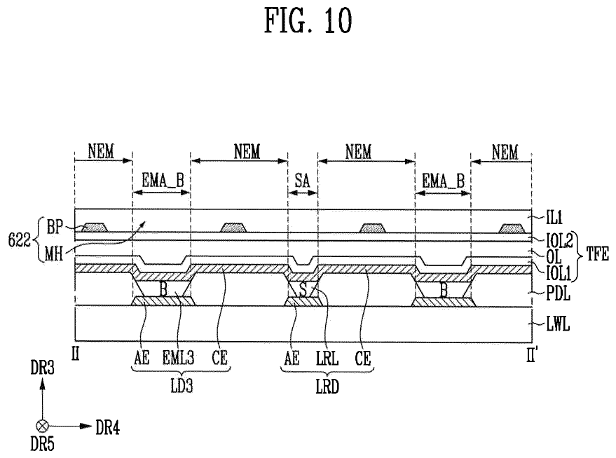

FIG. 8 mainly describes a cross-sectional view of the first sub-pixel SP1 and the second sub-pixel SP2. A cross-sectional view of the third sub-pixel SP3 and the photo sensor PHS of FIG. 7 may be understood through the description of FIG. 10. Accordingly, in FIG. 8, a cross-sectional view of a first light-emitting element LD1 which is a light-emitting element of the first sub-pixel SP1 and a second light-emitting element LD2 which is a light-emitting element of the second sub-pixel SP2 will be mainly described.

Referring to FIG. 8, a pixel defining layer PDL, light-emitting elements LD1 and LD2, an encapsulation layer TFE, a first sensing cell 612, and a first insulating layer IL1 may be positioned on a lower layer LWL. The lower layer LWL may include the above-described base substrate BSL (see FIGS. 3A and 3B) and at least a part of the element layer DSL (see FIG. 3A) positioned on the base substrate BSL. The light-emitting elements LD1 and LD2 may include the remaining part of the element layer DSL described above. The first sensing cell 612 and the first insulating layer IL1 may be included in the touch panel 210 described above (see FIG. 2).

In the light-emitting elements LD1 and LD2, the first light-emitting element LD1 of the above-described first sub-pixel SP1 (see FIG. 7) and the second light-emitting element LD2 of the second sub-pixel SP2 (see FIG. 7) are exemplarily shown. The first light-emitting element LD1 may include an anode electrode AE, a first light-emitting layer EML1, and a cathode electrode CE. The second light-emitting element LD2 may include an anode electrode AE, a second light-emitting layer EML2, and a cathode electrode CE.

The anode electrode AE may be disposed for each sub-pixel. The pixel defining layer PDL exposing the anode electrode AE may be disposed on the anode electrode AE. The anode electrode AE may include a metal layer such as silver (Ag), magnesium (Mg), aluminum (Al), platinum (Pt), palladium (Pd), gold (Au), nickel (Ni), neodymium (Nd), iridium (Ir), chromium (Cr), an alloy thereof, and/or indium tin oxide (ITO), indium zinc oxide (IZO), zinc oxide (ZnO) or indium tin zinc oxide (ITZO).

The pixel defining layer PDL may be positioned to overlap the non-emission area NEM. The pixel defining layer PDL may include an organic insulating layer including an organic material. As the organic material, one or more among an acryl resin, an epoxy resin, a phenol resin, a polyamide resin, and a polyimide resin may be selected. The pixel defining layer PDL may include a light absorbing material. The pixel defining layer PDL may be coated with a light absorber to absorb light introduced from the outside. In an embodiment, the pixel defining layer PDL may include a carbon-based black pigment. In an embodiment, the pixel defining layer PDL may include an opaque metal material such as chromium (Cr), molybdenum (Mo), an alloy (MoTi) of molybdenum (Mo) and titanium (Ti), tungsten (W), vanadium (V), niobium (Nb), tantalum (Ta), manganese (Mn), cobalt (Co), or nickel (Ni) having a high light absorption rate. However, embodiments of the present disclosure are not limited thereto.

The light-emitting layers EML1 and EML2 may be disposed to overlap an area where at least a part of the pixel defining layer PDL is removed and the anode electrode AE is exposed. The light-emitting layers EML1 and EML2 may include an organic light-emitting layer. Depending on the organic material included in the light-emitting layers EML1 and EML2, the light-emitting layers EML1 and EML2 may emit light of different wavelength bands. For example, the first emission layer EML1 may emit light in a first wavelength band (e.g., a red wavelength band). For example, the second light-emitting layer EML2 may emit light in a second wavelength band (e.g., a green wavelength band). An area where the first light-emitting layer EML1 is located may correspond to the first emission area EMA_R. An area where the second light-emitting layer EML2 is located may correspond to the second emission area EMA_G.

The cathode electrode CE may be disposed on the light-emitting layers EML1 and EML2 (e.g., in the third direction DR3). The cathode electrode CE may be deposited (e.g., entirely deposited) in the emission areas EMA_R and EMA_G and the non-emission area NEM. The cathode electrode CE may include a metal layer such as silver (Ag), magnesium (Mg), aluminum (Al), platinum (Pt), palladium (Pd), gold (Au), nickel (Ni), neodymium (Nd), iridium (Ir), or chromium (Cr), and/or a light-transmitting conductive layer such as ITO, IZO, ZnO, or ITZO. For example, the cathode electrode CE may consist of a multilayer of two or more layers including a thin metal layer. For example, the cathode electrode CE may include a triple layer of ITO/AG/ITO.

In an embodiment, the light-emitting elements LD1 and LD2 may further include a hole transport layer disposed between the anode electrode AE and the light-emitting layers EML1 and EML2. In an embodiment, the light-emitting elements LD1 and LD2 may further include an electron transport layer disposed between the cathode electrode CE and the light-emitting layers EML1 and EML2.

The encapsulation layer TFE may be located on the cathode electrode CE (e.g., in the third direction DR3). The encapsulation layer TFE may have a laminated structure formed of an inorganic material, an organic material, and an inorganic material in that order. Referring to FIG. 8, the encapsulation layer TFE may be formed by sequentially depositing a first inorganic encapsulation layer IOL1 including an inorganic material, an organic encapsulation layer OL including an organic material, and a second inorganic encapsulation layer IOL2 including an inorganic material. The encapsulation layer TFE may protect the light-emitting elements LD1 and LD2 (e.g., the light-emitting layers EML1 and EML2) under the encapsulation layer TFE from external moisture and/or oxygen.

The body portion BP of the first sensing cell 612 may be disposed on the encapsulation layer TFE. The first insulating layer IL1 may be positioned on the body portion BP of the first sensing cell 612.

The first insulating layer IL1 may insulate the touch electrodes from each other. Referring further to FIG. 5, the plurality of first touch electrodes Tx1 to Txa may be insulated from each other by the first insulating layer IL1. The plurality of second touch electrodes Rx1 to Rxb may be insulated from each other by the first insulating layer IL1. Referring further to FIG. 6, the first connection electrode 614 and the second connection electrode 624 may be insulated from each other by the first insulating layer IL1. For example, the first connection electrode 614 and the second connection electrode 624 may be positioned to overlap each other in the thickness direction (e.g., the third direction DR3) with the first insulating layer IL1 interposed therebetween.

The mesh hole MH of the first sensing cell 612 may be positioned to overlap the first and second emission areas EMA_R and EMA_G.

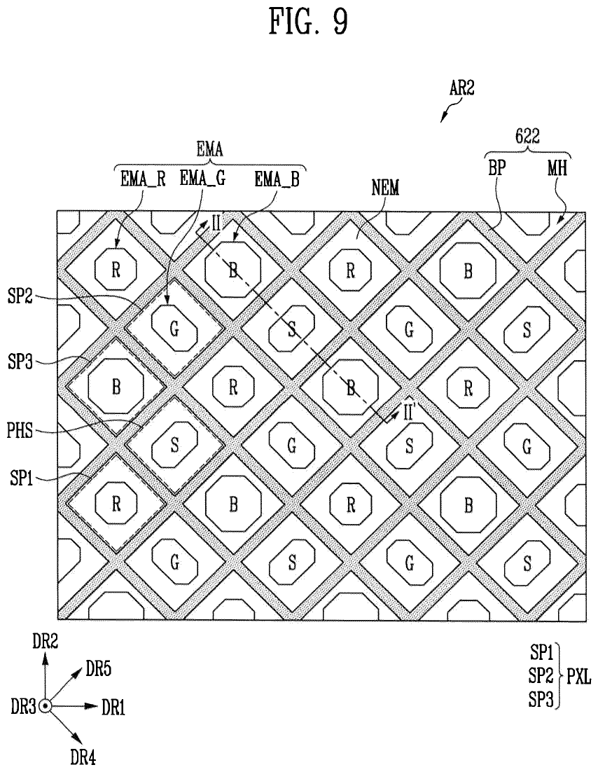

FIG. 9 is an enlarged view of a second area AR2 of FIG. 6.

Comparing FIGS. 9 and 7, the description of the first sensing cell 612 may be applied as a description of the second sensing cell 622 as a whole. The second sensing cell 622 may include a body portion BP and a mesh hole MH. The description of the remaining configurations is as described above with reference to FIG. 7.

FIG. 10 is a cross-sectional view taken along line II-II′ of FIG. 9.

FIG. 10 mainly describes a cross-sectional view of the third sub-pixel SP3 and the photo sensor PHS. A cross-sectional view of the first sub-pixel SP1 and the second sub-pixel SP2 of FIG. 9 may be understood through the description of FIG. 8 described above. Accordingly, in FIG. 10, a cross-sectional view of a third light-emitting element LD3 which is a light-emitting element of the third sub-pixel SP3 and a light-receiving element LRD of the photo sensor PHS will be mainly described.

Referring to FIG. 10, a pixel defining layer PDL, a third light-emitting element LD3, a light-receiving element LRD, an encapsulation layer TFE, a second sensing cell 622, and a first insulating layer IL1 may be positioned on a lower layer LWL. The lower layer LWL may include the above-described base substrate BSL (see FIGS. 3A and 3B) and at least a part of the element layer DSL (see FIG. 3A) positioned on the base substrate BSL. The third light-emitting element LD3 and the light-receiving element LRD may include the remaining part of the element layer DSL described above. The second sensing cell 622 and the first insulating layer IL1 may be included in the touch panel 210 described above (see FIG. 2).

The third light-emitting element LD3 may include an anode electrode AE, a third light-emitting layer EML3, and a cathode electrode CE. The light-receiving element LRD may include an anode electrode AE, a light-receiving layer LRL, and a cathode electrode CE.

The anode electrode AE may be disposed for each sub-pixel and each photo sensor. A pixel defining layer PDL exposing the anode electrode AE may be disposed on the anode electrode AE.

The pixel defining layer PDL may be positioned to overlap the non-emission area NEM. The non-emission area NEM may include a peripheral area of each of the third emission area EMA_B and a light-receiving area SA. The pixel defining layer PDL may include an organic insulating layer including an organic material. The pixel defining layer PDL may include a light absorbing material. However, embodiments of the present disclosure are not limited thereto.

The third light-emitting layer EML3 may be disposed to overlap an area where at least a part of the pixel defining layer PDL is removed and the anode electrode AE is exposed. The third light-emitting layer EML3 may include an organic light-emitting layer. For example, the third light-emitting layer EML3 may emit light in a third wavelength band (e.g., a blue wavelength band). An area where the third light-emitting layer EML3 is located may correspond to the third emission area EMA_B.

The light-receiving layer LRL may be disposed to overlap an area where at least a part of the pixel defining layer PDL is removed and the anode electrode AE is exposed. An area where the light-receiving layer LRL is located may correspond to the light-receiving area SA.

The cathode electrode CE may be disposed on the third light-emitting layer EML3 and the light-receiving layer LRL (e.g., in the third direction DR3 thereof). The cathode electrode CE may be deposited (e.g., entirely deposited) in the third emission area EMA_B, the light-receiving area SA, and the non-emission area NEM. The cathode electrode CE may include a light-transmitting conductive layer. For example, the cathode electrode CE may consist of a multilayer of two or more layers including a thin metal layer. For example, the cathode electrode CE may include a triple layer of ITO/Ag/ITO.

In an embodiment, the third light-emitting element LD3 may further include a hole transport layer disposed between the anode electrode AE and the third light-emitting layer EML3. In an embodiment, the third light-emitting element LD3 may further include an electron transport layer disposed between the cathode electrode CE and the third light-emitting Layer EML3.

The encapsulation layer TFE may be located on the cathode electrode CE (e.g., in the third direction DR3). The encapsulation layer TFE may have a laminated structure formed of an inorganic material, an organic material, and an inorganic material in that order. Referring to FIG. 10, the encapsulation layer TFE may be formed by sequentially depositing a first inorganic encapsulation layer IOL1 containing an inorganic material, an organic encapsulation layer OL containing an organic material, and a second inorganic encapsulation layer IOL2 containing an inorganic material. The encapsulation layer TFE may protect the lower third light-emitting element LD3 (e.g., the third light-emitting layer EML3) and the light-receiving element LRD (e.g., a light-receiving layer LRL) from external moisture and/or oxygen.

The body portion BP of the second sensing cell 622 may be disposed on the encapsulation layer TFE. The first insulating layer IL1 may be positioned on the body portion BP of the second sensing cell 622.

The mesh hole MH of the second sensing cell 622 may be positioned to overlap the third emission area EMA_B and the light-receiving area SA.

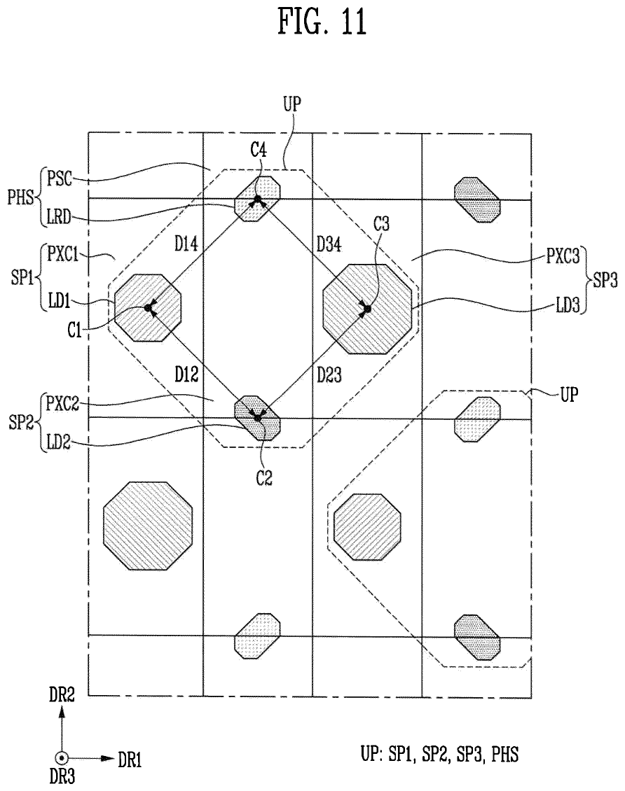

FIG. 11 is a layout diagram illustrating a display area DA according to an embodiment.

Referring to FIG. 11, the display area DA may include sub-pixels SP1, SP2, and SP3 and a photo sensor PHS. The sub-pixels SP1, SP2, and SP3 and the photo sensor PHS may be included in one unit pixel UP.

The unit pixel UP may be defined as a minimum unit of pixels capable of displaying a white image. The unit pixel UP may sense light. In an embodiment, the unit pixels UP may be alternately arranged in a zigzag shape in a plan view. For example, the unit pixels UP may be arranged in a diagonal direction. For example, the unit pixels UP may be alternately arranged in the form of horizontal or vertical stripes in a plan view.

The first sub-pixel SP1 may include a first light-emitting element LD1 for emitting light in a first wavelength band (e.g., a red wavelength band), and a first pixel driving circuit PXC1 for applying a driving current to the first light-emitting elements LD1.

The second sub-pixel SP2 may include a second light-emitting element LD2 for emitting light in a second wavelength band (e.g., a green wavelength band), and a second pixel driving circuit PXC2 for applying a driving current to the second light-emitting elements LD2.

The third sub-pixel SP3 may include a third light-emitting element LD3 for emitting light in a third wavelength band (e.g., a blue wavelength band), and a third pixel driving circuit PXC3 for applying a driving current to the third light-emitting elements LD3.

The photo sensor PHS may include a light-receiving element LRD for detecting light (e.g., light of a predetermined wavelength band) and a sensor circuit PSC connected to the light-receiver element LRD.

Referring to FIG. 11, a distance between a center C1 of the first light-emitting element LD1 and a center C2 of the second light-emitting element LD2 adjacent to each other in the unit pixel UP may be a first distance D12. A distance between the center C2 of the second light-emitting element LD2 and a center C3 of the third light-emitting element LD3 adjacent to each other in the unit pixel UP may be a second distance D23. The distance between the center C3 of the third light-emitting element LD3 and a center C4 of the light-receiving element LRD adjacent to each other in the unit pixel UP may be a third distance D34. The distance between the center C4 of the light-receiving elements LRD adjacent to each other in the unit pixel UP and the first light-emitting element LD1 may be a fourth distance D14. In an embodiment, the first distance D12, the second distance D23, the third distance D34, and the fourth distance D14 may each be substantially the same. However, embodiments of the present disclosure are not limited thereto.

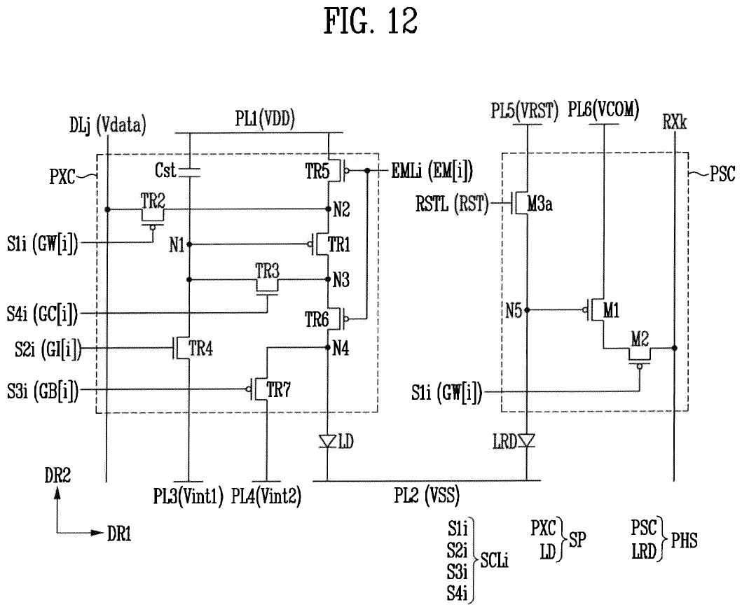

FIG. 12 is an equivalent circuit diagram of a sub-pixel SP and a photo sensor PHS according to an embodiment.

Referring to FIG. 12, the sub-pixel SP may include a pixel driving circuit PXC and a light-emitting element LD. The photo sensor PHS may include a sensor circuit PSC and a light-receiving element LRD.

The pixel driving circuit PXC may be configured to control a magnitude of a current flowing through the light-emitting element LD. The pixel driving circuit PXC may include at least one switching element (e.g., a transistor) and one or more storage elements (e.g., capacitors).

Referring to FIG. 12, the pixel driving circuit PXC may include first to seventh pixel transistors TR1 to TR7 and a capacitor Cst.

The pixel driving circuit PXC may be connected to an i-th scan line SCLi (hereinafter, also referred to as a scan line SCLi), an i-th light emission control line EMLi (hereinafter, referred to as a light emission control line EMLi), and a j-th data line DLj (hereinafter, referred to as a data line DLj).

The scan line SCLi may include first to fourth scan lines S1i, S2i, S3i, and S4i.

The first pixel transistor TR1 may be configured to adjust a magnitude of a current flowing between a second node N2 and a third node N3 in response to a voltage of a first node N1. The first node N1 may be electrically connected to a gate electrode of the first pixel transistor TR1. The second node N2 may be electrically connected to a first electrode (e.g., any one of a source electrode and a drain electrode) of the first pixel transistor TR1. The third node N3 may be electrically connected to a second electrode (e.g., another one of a source electrode and a drain electrode) of the first pixel transistor TR1. The first pixel transistor TR1 may be referred to as a driving transistor.

The second pixel transistor TR2 may be configured to switch an electrical connection between the second node N2 and the data line DLj. The second pixel transistor TR2 may include a gate electrode connected to the first scan line S1i. The second pixel transistor TR2 may be configured to transfer a voltage (e.g., a data voltage) applied to the data line DLj to the second node N2 in response to a first scan signal GW[i] of a turn-on level. The first scan signal GW[i] may be applied to the first scan line S1i. The second pixel transistor TR2 may be referred to as a switching transistor.

The third pixel transistor TR3 may be configured to switch an electrical connection between the first node N1 and the third node N3. The third pixel transistor TR3 may include a gate electrode connected to the fourth scan line S4i. The third pixel transistor TR3 may be configured to switch an electrical connection between the first node N1 and the third node N3 in response to a fourth scan signal GC[i]. The fourth scan signal GC[i] may be applied to the fourth scan line S4i. When the third pixel transistor TR3 is turned on, the first pixel transistor TR1 may be connected in the form of a diode. The third pixel transistor TR3 may be referred to as a compensation transistor.

The fourth pixel transistor TR4 may be configured to switch an electrical connection between the first node N1 and a third power line PL3. The fourth pixel transistor TR4 may include a gate electrode connected to the second scan line S2i. The fourth pixel transistor TR4 may be configured to switch an electrical connection between the first node N1 and the third power line PL3 in response to a second scan signal GI[i]. The second scan signal GI[i] may be applied to the second scan line S2i. A first initialization voltage Vint1 may be applied to the third power line PL3. When the fourth pixel transistor TR4 is turned on, the voltage of the first node N1 may be initialized to the first initialization voltage Vint1. The fourth pixel transistor TR4 may be referred to as a first initialization transistor.

The fifth pixel transistor TR5 may be configured to switch an electrical connection between the second node N2 and a first power line PL1. The fifth pixel transistor TR5 may include a gate electrode connected to the light emission control line EMLi. The fifth pixel transistor TR5 may be configured to switch an electrical connection between the second node N2 and the first power line PL1 in response to a light emission control signal EM[i]. When the fifth pixel transistor TR5 is turned on, a first power voltage VDD may be applied to the second node N2. The fifth pixel transistor TR5 may be referred to as a first light emission control transistor.

The sixth pixel transistor TR6 may be configured to switch an electrical connection between the third node N3 and a fourth node N4. The sixth pixel transistor TR6 may include a gate electrode connected to the light emission control line EMLi. The sixth pixel transistor TR6 may be configured to switch an electrical connection between the third node N3 and the fourth node N4 in response to the light emission control signal EM[i]. Referring to FIG. 12, the sixth pixel transistor TR6 and the fifth pixel transistor TR5 may be electrically connected to the same emission control line EMLi. However, embodiments of the present disclosure are not limited thereto. The sixth pixel transistor TR6 may be referred to as a second light emission control transistor.

The seventh pixel transistor TR7 may be configured to switch an electrical connection between the fourth node N4 and a fourth power line PL4. The seventh pixel transistor TR7 may include a gate electrode connected to the third scan line S3i. The seventh pixel transistor TR7 may be configured to switch an electrical connection between the fourth node N4 and the fourth power line PL4 in response to a third scan signal GB[i]. When the seventh pixel transistor TR7 is turned on, the voltage of the fourth node N4 may be initialized to a second initialization voltage Vint2. The second initialization voltage Vint2 may be applied to the fourth power line PL4. The seventh pixel transistor TR7 may be referred to as a second initialization transistor.

The capacitor Cst may be configured to maintain a voltage of the first node N1. The capacitor Cst may be configured to supply a voltage to the first node N1 during one frame period. The capacitor Cst may include one electrode electrically connected to the first node N1 and the other electrode electrically connected to a power line (e.g., the first power line PL1). A data voltage Vdata (or a voltage corresponding to data voltage Vdata) may be applied to one electrode of the capacitor Cst. The capacitor Cst may supply the data voltage Vdata (or a voltage corresponding to the data voltage Vdata) to the first node N1 for one frame period. The capacitor Cst may be referred to as a storage capacitor.

Each of the first to seventh pixel transistors TR1 to TR7 may be formed of an N-type transistor or a P-type transistor.

In the N-type transistor, a voltage at a turn-on level may be a voltage at a high logic level, and a voltage at a turned-off level may be a power at a low logic level. In the P-type transistor, a voltage at a turn-on level may be a voltage at a low logic level, and a voltage at a turned-off level may be a power at a high logic level.

Referring to FIG. 12, among the first to seventh pixel transistors TR1 to TR7, the third pixel transistor TR3 and the fourth pixel transistor TR4 are shown to be N-type transistors, and the rest are shown to be P-type transistors. However, embodiments of the present disclosure are not limited thereto.

One or more of the first to seventh pixel transistors TR1 to TR7 may include an oxide semiconductor. One or more of the first to seventh pixel transistors TR1 to TR7 may include a silicon semiconductor (e.g., an amorphous silicon (a-Si) semiconductor or a low temperature polycrystalline silicon (LTPS) semiconductor). For example, the third pixel transistor TR3 and the fourth pixel transistor TR4 may include an oxide semiconductor. However, embodiments of the present disclosure are not limited thereto.

The light-emitting element LD may be connected between the fourth node N4 and a second power line PL2. The fourth node N4 may be electrically connected to the anode electrode of the light-emitting element LD. The second power line PL2 may be electrically connected to the cathode electrode of the light-emitting element LD. A second power voltage VSS may be applied to the second power line PL2.

The light-emitting element LD may include a light-emitting layer. Depending on the type of the light-emitting layer, the light-emitting element LD may be implemented as an organic light-emitting element including an organic light-emitting layer, an inorganic light-emitting element including a inorganic light-emitting layer or a quantum dot light-emitting element including quantum dots (e.g., nano-rods).

One pixel driving circuit PXC may be connected to two or more light-emitting elements LD. Two or more light-emitting elements LD may be connected in series and/or parallel to each other.

The sensor circuit PSC may include first to third sensor transistors M1, M2, and M3a.

The first sensor transistor M1 may be configured to switch an electrical connection between a sixth power line PL6 and the second sensor transistor M2. The first sensor transistor M1 may include a gate electrode connected to a fifth node N5. The first sensor transistor M1 may switch an electrical connection between a fifth power line PL5 and the second sensor transistor M2 according to a voltage level of the fifth node N5. A fourth power voltage VCOM may be applied to the sixth power line PL6.

The second sensor transistor M2 may be configured to switch an electrical connection between the first sensor transistor M1 and a k-th sensing line RXk (or a sensing line RXk). The second sensor transistor M2 may include a gate electrode connected to the first scan line S1i. The second sensor transistor M2 may be configured to switch an electrical connection between the first sensor transistor M1 and the sensing line RXk in response to a scan signal (e.g., the first scan signal GW[i]). The second sensor transistor M2 may include a gate electrode electrically connected to the first scan line S1i.

The third sensor transistor M3a may be configured to switch an electrical connection between the fifth power line PL5 and the fifth node N5. The third sensor transistor M3a may include a gate electrode electrically connected to a reset control line RSTL. The third sensor transistor M3a may be configured to switch an electrical connection between the fifth power line PL5 and the fifth node N5 in response to a reset signal RST input to the reset control line RSTL. The third sensor transistor M3a may include a gate electrode connected to the reset control line RSTL. When the third sensor transistor M3a is turned on, a voltage of the fifth node N5 may be initialized to a third power voltage VRST. Thereby, the third power voltage VRST may be applied to the light-receiving element LRD. The third power voltage VRST may be a turn-off level voltage of the first sensor transistor M1. The third power voltage VRST may be referred to as a reset voltage.

Each of the first to third sensor transistors M1, M2, and M3a may be a P-type transistor or an N-type transistor. In an embodiment, the first to third sensor transistors M1, M2, and M3a may each include any one of an amorphous silicon semiconductor, a low-temperature polysilicon semiconductor, and an oxide semiconductor.

Referring to FIG. 12, the third sensor transistor M3a may be an N-type transistor, and the first and second sensor transistors M1 and M2 may be P-type transistors. However, embodiments of the present disclosure are not limited thereto.

The light-receiving element LRD may provide a current flowing in a direction from the fifth node N5 to the second power line PL2 in response to an irradiated light. The light-receiving element LRD may be implemented as, for example, a photodiode. When a current flows through the light-receiving element LRD, the voltage of the fifth node N5 may be lowered to the second power voltage VSS.

In the embodiments of the present disclosure, a process in which a current flows through the sensing line RXk will be briefly described as follows.

In embodiments of the present disclosure, when light is irradiated to the light-receiving element LRD, a current may flow through the light-receiving element LRD. When a current flows through the light-receiving element LRD, the voltage of the fifth node N5 may gradually decrease. When the voltage of the fifth node N5 gradually decreases and becomes lower than a threshold voltage of the first sensor transistor M1, the first sensor transistors M1 may be turned on. When the first scan signal GW[i] of a turn-on level is input to the second sensor transistor M2, the second sensor transistors M2 may be turned on. Accordingly, when the first and second sensor transistors M1 and M2 are turned on, a current path from the sixth power line PL6 to the sensing line RXk may be formed. By integrating the current flowing through the sensing line RXk, the amount of light incident on the light-receiving element LRD may be calculated.

Embodiments of the present disclosure may use the photo sensor PHS to measure the amount of reflected light reflected from an object adjacent to the photo sensor PHS.

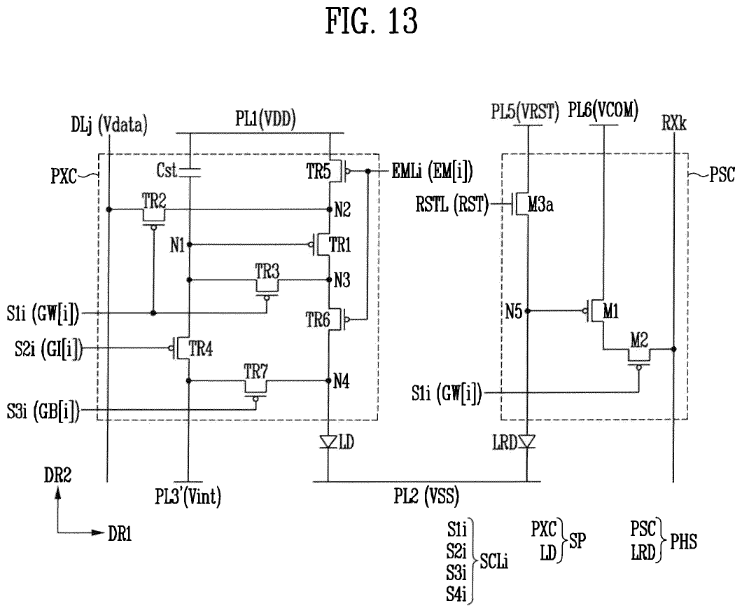

FIG. 13 is an equivalent circuit diagram of a sub-pixel SP and a photo sensor PHS according to another embodiment.

Compared with the equivalent circuit diagram of FIG. 13 and the equivalent circuit diagram in FIG. 12, in the equivalent circuit diagram shown in FIG. 13, the pixel driving circuit PXC may include the third pixel transistor TR3 and the fourth pixel transistor TR4 as P-type transistors.

The third pixel transistor TR3 may include a gate electrode electrically connected to the first scan line S1i. The third pixel transistor TR3 may be commonly connected to the first scan line S1i together with the second pixel transistor TR2.

The fourth pixel transistor TR4 may be configured to switch an electrical connection between a third power line PL3′ and the first node N1. An initialization voltage Vint may be applied to the third power line PL3′.

The seventh pixel transistor TR7 may be configured to switch an electrical connection between the third power line PL3′ and the fourth node N4.

According to the embodiment of FIG. 13, the number of scan lines and the number of power lines required are reduced compared to the embodiment of FIG. 12, and thus the circuit configuration may be simplified.

According to the embodiment of FIG. 12, by implementing the third pixel transistor TR3 and the fourth pixel transistor TR4 as N-type transistors compared to the embodiment of FIG. 13, it is possible to reduce leakage current and provide an image at a low frame rate.

Referring to FIGS. 12 and 13, the electronic device 100 (refer to FIG. 1) may acquire (or generate) a pattern of an object (e.g., a pattern of a fingerprint, etc.) adjacent to the photo sensor PHS by sensing the photo sensor PHS. Accordingly, the electronic device 100 according to embodiments of the present disclosure may provide a fingerprint authentication function.

In another embodiment, the electronic device 100 (see FIG. 1) may sense a change in the amount of light around the electronic device 100 by sensing the photo sensor PHS, and generate information associated with a blood vessel of a user using the electronic device 100 with reference to the sensed change in the amount of the light. The information related to the blood vessel may include, for example, at least one of cardiovascular health information, blood pressure, atrial rhythm, atrial fibrillation, heart rate, and respiratory rate.

In another embodiment, the electronic device 100 (see FIG. 1) may provide a function of sensing illuminance around the electronic device 100 by sensing the photo sensor PHS, and adaptively adjusting the luminance of the electronic device 100 according to the sensed illuminance.

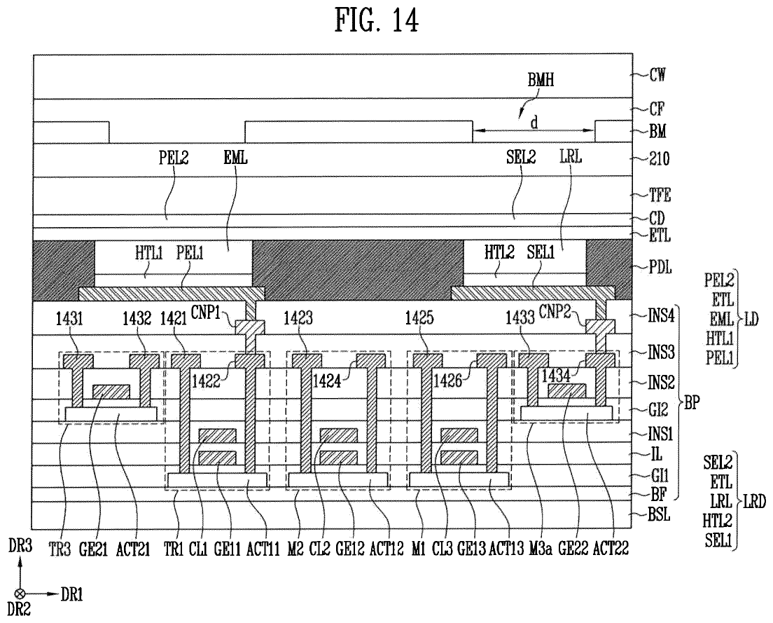

FIG. 14 is a diagram illustrating some of configurations of a sub-pixel and a photo sensor in a cross-sectional view of a display device according to embodiments of the present disclosure.

Referring to the foregoing drawings, pixel transistors TR1 to TR7 and sensor transistors M1, M2, and M3a may be included in a backplane structure BP of the display panel 220. The backplane structure may be disposed on the base substrate BSL.

FIG. 14 illustrates a first pixel transistor TR1, a third pixel transistor TR3, a first sensor transistor M1, a second sensor transistor M2, and a third sensor transistor M3a.

The base substrate BSL may include an insulating material such as glass or resin. In an embodiment, the base substrate BSL may include a material having flexibility to bend or fold. The base substrate BSL may have a single-layer structure or a multi-layer structure. In an embodiment, the base substrate BSL may include a first base layer, a first barrier layer, a second base layer and a second barrier layer sequentially stacked (e.g., sequentially stacked in the third direction DR3). The first base layer and the second base layer may include polyimide (PI), polyethersulfone (PES), polyarylate, polyetherimide (PEI), polyethylene naphthalate (PEN), polyethylene terephthalate (PET), polyphenylene sulfide (PPS), polycarbonate (PC), cellulose triacetate (CTA), and/or cellulose acetate propionate (CAP). The first barrier layer and the second barrier layer may include an inorganic insulator such as silicon oxide, silicon oxynitride, and/or silicon nitride. The base substrate BSL may be flexible.

A buffer layer BF may be disposed on the base substrate BSL. The buffer layer BF may prevent diffusion of impurities introduced from under the base substrate BSL. The buffer layer BF may be omitted depending on a material and process conditions of the base substrate BSL. The buffer layer BF may include an inorganic insulator such as silicon oxide, silicon oxynitride, or silicon nitride. The buffer layer BF may have a single-layer or multi-layer structure including the above-described materials.

First to third active patterns ACT11, ACT12, and ACT13 may be provided on the buffer layer BF. In an embodiment, the first to third active patterns ACT11, ACT12, and ACT13 may be formed of a polysilicon semiconductor. For example, the first to third active patterns ACT11, ACT12, and ACT13 may be formed through a low temperature polysilicon process (e.g., a low temperature polycrystalline silicon (LTPS) process).

A first gate insulating layer GI1 may be disposed on the first to third active patterns ACT11, ACT12, and ACT13. The first gate insulating layer GI1 may be an inorganic insulating layer including an inorganic material. The first gate insulating layer GI1 may include an inorganic insulating material such as silicon oxide (SiO2), silicon nitride (SiNₓ, where x is a positive number), silicon oxynitride (SiON), aluminum oxide (Al₂O₃), titanium oxide (TiO₂), tantalum oxide (Ta₂O₅), hafnium oxide (HfO₂), and/or zinc oxide (ZnO₂).

First to third gate electrodes GE11, GE12, and GE13 may be disposed on the first gate insulating layer GI1. The first gate electrode GE11 may overlap a channel area of the first active pattern ACT11 in the third direction DR3. The second gate electrode GE12 may overlap a channel area of the second active pattern ACT12 in the third direction DR3. The third gate electrode GE13 may overlap a channel area of the third active pattern ACT13 in the third direction DR3.

The first to third gate electrodes GE11, GE12, and GE13 may include a metal. For example, the first to third gate electrodes GE11, GE12, and GE13 may include at least one of a metal such as gold (Au), silver (Ag), aluminum (Al), molybdenum (Mo), chromium (Cr), titanium (Ti), nickel (Ni), neodymium (Nd), copper (Cu), or an alloy of metals. In addition, the first to third gate electrodes GE11, GE12, and GE13 may be formed of a single layer or may be formed of multiple layers in which two or more of metals and alloys are stacked.

An interlayer insulating layer IL may be disposed on the first to third gate electrodes GE11, GE12, and GE13. The interlayer insulating layer IL may be an inorganic insulating layer containing an inorganic material. As the inorganic material, polysiloxane, silicon nitride, silicon oxide or silicon oxynitride may be used.

Conductive patterns CL1, CL2, and CL3 may be disposed on the interlayer insulating layer IL. Referring further to FIGS. 12 and 13, the conductive patterns CL1, CL2, and CL3 may constitute at least one of an electrode of a capacitor Cst, scan lines (e.g., first to fourth scan lines S1i to S4i), a reset control line RSTL, a data line DLj, and a sensing line RXk, and power lines PL1 to PL6. The conductive patterns CL1, CL2, and CL3 may include at least one of a metal such as gold (Au), silver (Ag), aluminum (Al), molybdenum (Mo), chromium (Cr), titanium (Ti), nickel (Ni), neodymium (Nd), or copper (Cu), or an alloy of metals. The conductive patterns CL1, CL2, and CL3 may be formed of a single layer, or may be formed of multiple layers in which two or more of metals and alloys are stacked.

A first insulating layer INS1 may be disposed on the conductive patterns CL1, CL2, and CL3. The first insulating layer INS1 may be an inorganic insulating layer including an inorganic material. As the inorganic material, polysiloxane, silicon nitride, silicon oxide or silicon oxynitride may be used.

A fourth active pattern ACT21 and a fifth active pattern ACT22 may be disposed on the first insulating layer INS1. In an embodiment, the fourth and fifth active patterns ACT21 and ACT22 may include an oxide semiconductor. For example, the fourth and fifth active patterns ACT21 and ACT22 may be formed through a metal oxide semiconductor formation process.

A second gate insulating layer GI2 may be disposed on the fourth active pattern ACT21 and the fifth active pattern ACT22. The second gate insulating layer GI2 may be an inorganic insulating layer including an inorganic material. For example, one or more of polysiloxane, silicon nitride, silicon oxide, and silicon oxynitride may be selected as the inorganic material.

Fourth and fifth gate electrodes GE21 and GE22 may be disposed on the second gate insulating layer GI2. The fourth gate electrode GE21 may overlap a channel area of the fourth active pattern ACT21 in the third direction DR3. The fifth gate electrode GE22 may overlap a channel area of the fifth active pattern ACT22 in the third direction DR3. The fourth and fifth gate electrodes GE21 and GE22 may include a metal. For example, the fourth and fifth gate electrodes GE21 and GE22 may include at least one of a metal such as gold (Au), silver (Ag), aluminum (Al), molybdenum (Mo), chromium (Cr), titanium (Ti), nickel (Ni), neodymium (Nd), copper (Cu), or an alloy of metals. In addition, the fourth and fifth gate electrodes GE21 and GE22 may be formed of a single layer or may be formed of multiple layers in which two or more of metals and alloys are stacked.

A second insulating layer INS2 may be disposed on the fourth and fifth gate electrodes GE21 and GE22. For example, the second insulating layer INS2 may be an inorganic insulating layer including an inorganic material. For example, one or more of polysiloxane, silicon nitride, silicon oxide, and silicon oxynitride may be selected as the inorganic material.

First source/drain electrodes 1421 and 1422, second source/drain electrode 1423 and 1424, third source/drain electrode 1425 and 1426, fourth source/drain electrodes 1431 and 1432, and fifth source/drain electrons 1433 and 1434 may be disposed on the second insulating layer INS2. The first to fifth source/drain electrodes 1421, 1422, 1423, 1424, 1425, 1426, 1431, 1432, 1433, and 1434 may be connected to corresponding first to fifth active patterns ACT11, ACT12, ACT13, ACT21, and ACT22 through contact holes, respectively.

The first to fifth source/drain electrodes 1421, 1422, 1423, 1424, 1425, 1426, 1431, 1432, 1433, and 1434 may include a metal. The first to fifth source/drain electrodes 1421, 1422, 1423, 1424, 1425, 1426, 1431, 1432, 1433, and 1434 may include a material having excellent conductivity. For example, the first to fifth source/drain electrodes 1421, 1422, 1423, 1424, 1425, 1426, 1431, 1432, 1433, and 1434 may include a conductive material including molybdenum (Mo), aluminum (Al), copper (Cu) and titanium (Ti). The first to fifth source/drain electrodes 1421, 1422, 1423, 1424, 1425, 1426, 1431, 1432, 1433, and 1434 may be formed of a single-layer structure or a multi-layer structure including the above material. For example, the first to fifth source/drain electrodes 1421, 1422, 1423, 1424, 1425, 1426, 1431, 1432, 1433, and 1434 may have a multilayer structure of Ti/Al/Ti.

A third insulating layer INS3 may be disposed on the first to fifth source/drain electrodes 1421, 1422, 1423, 1424, 1425, 1426, 1431, 1432, 1433, and 1434. For example, the third insulating layer INS3 may be an organic insulating layer including an organic material. The third insulating layer INS3 may include an organic insulating material such as a general-purpose polymer such as polymethylmethacrylate or polystyrene, a polymer derivative having a phenolic group, an acrylic polymer, an imide polymer, an aryl ether polymer, an amide polymer, a fluorine polymer, a p-xylene polymer, a vinyl alcohol polymer, or a blend thereof. The third insulating layer INS3 may planarize an area on the first to fifth source/drain electrodes 1421, 1422, 1423, 1424, 1425, 1426, 1431, 1432, 1433, and 1434.

Connection patterns CNP1 and CNP2 may be disposed on the third insulating layer INS3. The first connection pattern CNP1 may be connected to the first drain electrode 1422 through a contact hole penetrating the third insulating layer INS3. The second connection pattern CNP2 may be connected to the fifth source electrode 1434 through a contact hole penetrating the third insulating layer INS3.

The connection patterns CNP1 and CNP2 may be made of at least one of metals such as gold (Au), silver (Ag), aluminum (Al), molybdenum (Mo), chromium (Cr), titanium (Ti), nickel (Ni), neodymium (Nd), copper (Cu), or an alloy of metals.

A fourth insulating layer INS4 may be disposed on the connection patterns CNP1 and CNP2. The fourth insulating layer INS4 may be an organic insulating layer including an organic material. The fourth insulating layer INS4 may include an organic insulating material such as a general-purpose polymer such as polymethylmethacrylate or polystyrene, a polymer derivative having a phenolic group, an acrylic polymer, an imide polymer, an aryl ether polymer, an amide polymer, a fluorine polymer, a p-xylene polymer, a vinyl alcohol polymer, or a blend thereof. The fourth insulating layer INS4 may planarize areas on the connection patterns CNP1 and CNP2.

A pixel layer including a first pixel electrode PEL1, a first sensor electrode SEL1, and a bank layer BK may be provided on the fourth insulating layer INS4.

The pixel layer may include a light-emitting element LD connected to a pixel driving circuit (e.g., the pixel driving circuit PXC of FIGS. 12 and 13) and a light-receiving element LRD connected to a sensor circuit (e.g., the sensor circuit PSC of FIG. 12 or 13).

In an embodiment, the light-emitting element LD may include a first pixel electrode PEL1, a hole transport layer HTL1, a light-emitting layer EML, an electron transport layer ETL, and a second pixel electrode PEL2. In an embodiment, the light-receiving element LRD may include a first sensor electrode SEL1, a second hole transport layer HTL2, a light-receiving layer LRL, an electron transport layer ETL, and a second sensor electrode SEL2.

In an embodiment, the first pixel electrode PEL1 and the first sensor electrode SEL1 may include metal layers such as silver (Ag), magnesium (Mg), aluminum (Al), platinum (Pt), palladium (Pd), gold (Au), nickel (Ni), neodymium (Nd), iridium (Ir), chromium (Cr), alloys thereof, and/or indium tin oxide (ITO), indium zinc oxide (IZO), zinc oxide (ZnO) and indium tin zinc oxide (ITZO). The first pixel electrode PEL1 may be connected to the first drain electrode 1422 through a contact hole. The first sensor electrode SEL1 may be connected to the fifth source electrode 1434 through a contact hole.

The first pixel electrode PEL1 and the first sensor electrode SEL1 may be formed in the same process through patterning using a mask.

The bank layer (or a pixel defining layer) PDL that divides the emission area and the light-receiving area may be disposed on the fourth insulating layer INS4 on which the first pixel electrode PEL1 and the first sensor electrode SEL1 are formed.

The pixel defining layer PDL may include openings corresponding to the emission area and the light-receiving area.

A first hole transport layer HTL1 may be provided on an upper surface of the first pixel electrode PEL1 exposed by the pixel defining layer PDL. A second hole transport layer HTL2 may be disposed on an upper surface of the first sensor electrode SEL1 which is exposed. a hole may move to the light-emitting layer EML through the first hole transport layer HTL1, and a hole may move to a light-receiving layer LRL through the second hole transport layer HLT2.

Depending on the materials of the light-emitting layer EML and the light-receiving layer LRL, the first hole transport layer HTL1 and the second hole transport layer HLT2 may be the same or different.

The light-emitting layer EML may be disposed on the first hole transport layer HTL1. In an embodiment, the light-emitting layer EML may include an organic light-emitting layer. Depending on the organic material included in the light-emitting layer EML, the light-emitting layer EML may emit light in a red wavelength band, light in a green wavelength band, or light in a blue wavelength band. However, embodiments of the present disclosure are not limited thereto.

In an embodiment, an electron blocking layer may be disposed on the second hole transport layer HTL2 in the light-receiving area. The electron blocking layer may prevent a charge of the light-receiving layer LRL from moving to the second hole transport layer HTL2. In another embodiment, the electron blocking layer may be omitted.

The light-receiving layer LRL may be disposed on the second hole transport layer HTL2. The light-receiving layer LRL may emit electrons corresponding to light of a specific wavelength band. Thereby, the intensity (or amount) of light may be sensed.

In an embodiment, the light-receiving layer LRL may include a low-molecular-weight organic material. The low-molecular-weight organic material may include a phthalocyanine compound comprising at least one metal selected from the group consisting of copper (Cu), iron (Fe), nickel (Ni), cobalt (Co), manganese (Mn), aluminum (Al), palladium (Pd), tin (Sn), indium (In), lead (Pb), titanium (Ti), rubidium (Rb), vanadium (V), gallium (Ga), terbium (Tb), cerium (Ce), lanthanum (La) and zinc (Zn).

In an embodiment, the light-receiving layer LRL may be composed of two layers (or bi-layers). In an embodiment, the light-receiving layer LRL may include a layer including a phthalocyanine compound and a layer including C60. In an embodiment, the light-receiving layer LRL may include one mixing layer in which a phthalocyanine compound and C60 are mixed. However, this is exemplary, and the light-receiving layer LRL may include a polymer organic layer.

In an embodiment, a light detection band of the light-receiving element LRD may be determined according to a selection of the metal component of the phthalocyanine compound included in the light-receiving layer LRL. For example, in a case of a phthalocyanine compound comprising copper, visible light wavelengths in a range of approximately 600 to 800 nm may be absorbed. In a case of a phthalocyanine compound comprising tin (Sn), near-infrared wavelengths in a range of approximately 800 Nm to 1000 nm may be absorbed. Depending on the selection of the metal included in the phthalocyanine compound, a photo sensor capable of detecting a wavelength in a band desired by a user may be implemented. For example, the light-receiving layer LRL may be formed to selectively absorb light in a red wavelength band, light in a green wavelength band, or light in a blue wavelength band.

In an embodiment, an area of the light-receiving area may be smaller than an area of the emission area. The area of the light-receiving area may correspond to a width d of an opening BMH.

The second pixel electrode PEL2 and the second sensor electrode SEL2 may be disposed on the electron transport layer ETL. The second pixel electrode PEL2 and the second sensor electrode SEL2 may be provided as a common electrode CD integrally formed in the display area. Referring further to FIGS. 12 and 13, a second power voltage VSS may be supplied to the second pixel electrode PEL2 and the second sensor electrode SEL2. The common electrode CD may be formed of a metal layer such as silver (Ag), magnesium (Mg), aluminum (Al), platinum (Pt), palladium (Pd), gold (Au), nickel (Ni), neodymium (Nd), iridium (Ir), or chromium (Cr), and/or a transparent conductive layer such as ITO, IZO, ZnO, or ITZO. In an Embodiment, the common electrode CD may be formed of a multilayer of a double layer or more including a thin metal layer. For example, the common electrode CD may consist of a triple layer of ITO/AG/ITO.

An encapsulation layer TFE may be provided on the common electrode CD including the second pixel electrode PEL2 and the second sensor electrode SEL2. In an embodiment, the encapsulation layer TFE may have a laminated structure formed of an inorganic material, an organic material, and an inorganic material in that order. The top layer of the encapsulation layer TFE may include an inorganic material.

The touch panel 210 may be disposed on the encapsulation layer TFE. The touch panel 210 may include a touch electrode configured to sense a user's touch. In an embodiment, the touch panel 210 may be implemented in a self-capacitance manner. In another embodiment, the touch panel 210 may be implemented in a mutual capacitive manner.

A black matrix BM may be disposed on the touch panel 210. According to an embodiment, a color filter CF may be further disposed on the black matrix BM. In another embodiment, the color filter CF may be omitted. A cover window CW may be disposed on an uppermost layer of the display panel (e.g., an upper layer of the color filter CF).

At least a portion of the black matrix BM may overlap the pixel defining layer PDL. The black matrix BM may distinguish each sub-pixel so that light is not mixed between a plurality of sub-pixels. The black matrix BM may function as an optical system that collects light in the light-receiving element LRD. For example, referring to FIG. 12, at least a portion of the black matrix BM may be removed from an area corresponding to an opening BMH. The opening BMH may be positioned to overlap the light-receiving element LRD. The opening BMH allows the black matrix BM to function like an optical system.

The amount of light incident in a direction to the light-receiving element LRD (e.g., reflected light emitted from the light-emitting element LD and reflected by an external object) may be adjusted by the optical system formed by the black matrix BM (e.g., the width d of the optical system).

The electronic device according to embodiments of the present disclosure may sense photo sensors at different sensing resolutions in a mode for fingerprint authentication and in a mode for measuring bio-signal sensing data (or bio-signal).

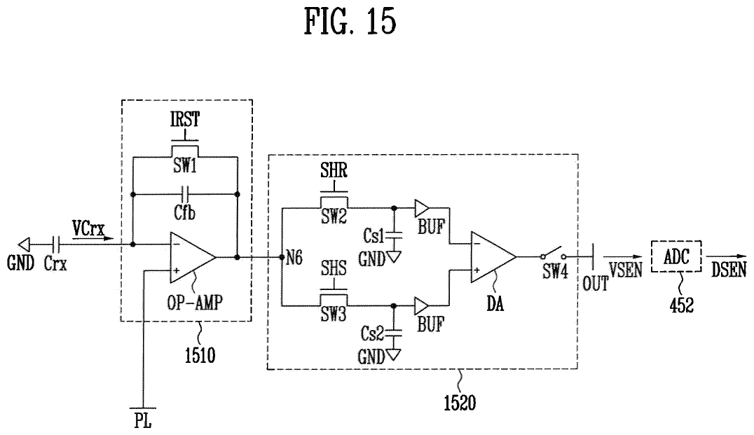

FIG. 15 is an equivalent circuit diagram of a readout circuit 450 (see FIG. 4) according to embodiments of the present disclosure.

Referring to FIG. 15, a readout circuit 450 (see FIG. 4) according to embodiments of the present disclosure may include an integrator 1510 and a sample and hold circuit (or also referred to as correlated double sampling circuit) 1520.

The integrator 1510 may include an operational amplifier OP-AMP, a feedback capacitor Cfb, and a first switching element SW1. An operation timing of the first switching element SW1 may be controlled by an integrator reset signal IRST.

The operational amplifier OP-AMP may include a first input terminal (e.g., “(−)” input terminal), a second input terminal (e.g., “(+)” input terminal), and an output terminal. The first input terminal may be an inverted input terminal.

The second input terminal may be a non-inverted input terminal. The first input terminal of the operational amplifier OP-AMP may be electrically connected to a sensing line RX. In an embodiment, a multiplexer may be further disposed between the first input terminal of the operational amplifier OP-AMP and the sensing line RX. A detection signal VCrx from a capacitor Crx corresponding to a voltage of the sensing line RX may be applied to the first input terminal of the operational amplifier OP-AMP.

The second input terminal of the operational amplifier OP-AMP may be electrically connected to a constant voltage line PL. In an embodiment, the second input terminal of the operational amplifier OP-AMP may be grounded GND.

The feedback capacitor Cfb may include one electrode connected to the first input terminal of the operational amplifier OP-AMP and the other electrode connected to the output terminal of the operational amplifier OP-AMP.

The first switching element SW1 may be configured to switch an electrical connection between the first input terminal and the output terminal of the operational amplifier OP-AMP. When the first switching element SW1 is turned on, a charge stored in the feedback capacitor Cfb may be discharged and the feedback capacitor Cfb may be reset.

The sample and hold circuit 1520 may include a second switching element SW2, a third switching element SW3, a first sampling capacitor Cs1, a second sampling capacitor Cs2, a differential amplifier (DA) and a fourth switching element SW4. The sample and hold circuit 1520 may include a sixth node N6 electrically connected to an output end of the integrator 1510 (e.g., an output end of an operational amplifier OP-AMP). The sample and hold circuit 1520 may be configured to store a value sensed by the photo sensor and output an analog voltage obtained by removing (or reducing) a noise component from the sensed value.

The second switching element SW2 may be configured to switch an electrical connection between the sixth node N6 and the first sampling capacitor Cs1. An operation timing of the second switching element SW2 may be controlled by a second sampling signal SHR.

The first sampling capacitor Cs1 may be configured to store a value corresponding to the noise component. The noise component may include, for example, a noise component basically present in the sensing line RX. The first sampling capacitor Cs1 may include one electrode electrically connected to the second switching element SW2 and the other electrode electrically connected to a constant voltage source (or a ground power source GND).

The third switching element SW3 may be configured to switch an electrical connection between the sixth node N6 and the second sampling capacitor Cs2. An operation timing of the third switching element SW3 may be controlled by a first sampling signal SHS.

The second sampling capacitor Cs2 may be configured to store a value sensed by the photo sensor including the noise component. The second sampling capacitor Cs2 may include one electrode electrically connected to the third switching element SW3 and the other electrode electrically connected to a constant voltage source (or a ground power source GND).

The differential amplifier may include a first input terminal (e.g., “(−)” input terminal), a second input terminal (e.g., “(+)” input terminal), and an output terminal. The differential amplifier may be configured to amplify and output a signal (e.g., a voltage) corresponding to a difference between a signal (e.g., a voltage) input to the second input terminal and a signal (e.g., a voltage) input to the first input terminal. The first input terminal may be an inverted input terminal. The second input terminal may be a non-inverted input terminal. The first input terminal of the differential amplifier may be configured to receive a voltage applied from the first sampling capacitor Cs1. In an embodiment, a buffer BUF may be further disposed between the first input terminal of the differential amplifier and the first sampling capacitor Cs1.

The second input terminal of the differential amplifier may be configured to receive a voltage applied from the second sampling capacitor Cs2. A buffer BUF may be further disposed between the second input terminal of the differential amplifier and the second sampling capacitor Cs2.

The fourth switching element SW4 may be configured to switch an electrical connection between an output end of the differential amplifier and an input end OUT of the analog-to-digital converter 452. When the fourth switching element SW4 is turned on, a sensing voltage VSEN corresponding to the amount of light received by the photo sensor may be input to the analog-to-digital converter 452.

The second switching element SW2 and the third switching element SW3 may correspond to switching elements for sampling signals. The fourth switching element SW4 may correspond to a switching element for holding a signal.

The analog-to-digital converter 452 may convert the input sensing voltage VSEN into a digital value and output a converted digital value DSEN.

FIG. 16 is a driving timing diagram for sensing a photo sensor at a first sensing resolution in case that the electronic device 100 (see FIG. 1) operates in a fingerprint authentication mode.

Referring to FIG. 16, a timing diagram of a reset signal RST, first scan signals GW[1] to GW[n], and a first sampling signal SHS is shown.

Referring further to FIGS. 4 and 15, the reset circuit 460 may output the reset signal RST. The scan driving circuit 420 may output the first scan signals GW[1] to GW[n]. The first sampling signal SHS is included in the readout circuit control signal RCS. The timing controller 440 may output the first sampling signal SHS. A timing at which the readout circuit 450 senses the photo sensor PHS may be controlled in response to the first sampling signal SHS. A length of a period during which the readout circuit 450 senses the photo sensor PHS may be controlled in response to the first sampling signal SHS.

When operating in the fingerprint authentication mode, a reset frame may be started. The reset circuit 460 (see FIG. 4) may output the reset signal RST in the reset frame. The reset signal RST may be output before the first scan signals GW[1] to GW[n]. Accordingly, the voltages of the sensing lines RX1 to RXk (see FIG. 4) may be reset (or initialized). Referring to FIGS. 12, 13, and 15 together, when the reset signal RST is input and the third sensor transistor M3a is turned on, the third power voltage VRST is applied to the fifth node N5. The first sensor transistor M1 may be turned on in response to the third power voltage VRST applied to the fifth node N5. The second sensor transistor M2 may be turned on in response to a corresponding one of the plurality of first scan signals GW[p] to GW[q] (where p, q being an integer of 1 or more). When the first sensor transistor M1 and the second sensor transistor M2 are turned on, a voltage level of the sensing line RXk may be initialized to the fourth power voltage VCOM.

After the reset frame, an exposure time (or exposure integration time EIT) may proceed. The exposure time may include one or more hold frames. The exposure time may correspond to a period in which the photo sensor receives light (e.g., a period in which light is received from after the reset frame to before the readout frame). Further referring to FIGS. 12, 13, and 15, the light sensor PHS may receive light during the exposure time so that a current may flow through the light-receiving element LRD. In the fingerprint authentication mode, the length of the exposure time may be set long enough that the magnitude of the current flowing through the light-receiving element LRD becomes relatively constant (or converges to a constant value). For example, the length of the exposure time may be about 100 milliseconds (ms) (or about 12 frame periods based on a scan rate of 120 Hz). However, embodiments of the present disclosure are not limited thereto. Accordingly, each of the plurality of photo sensors PHS may reflect a difference in the amount of light received.

After the exposure time, one or more readout frames may be performed. In the readout frame, a first sampling signal SHS of a turn-on level may be input to the readout circuit 450 (see FIG. 4). A plurality of photo sensors may be sensed in the readout frame. In the fingerprint authentication mode, the first sampling signal SHS of the turn-on level may be applied for one horizontal period.

Embodiments of the present disclosure may sense a plurality of photo sensors in one readout frame period in a fingerprint authentication mode. Embodiments of the present disclosure may selectively sense only a corresponding photo sensor of the plurality of photo sensors in each readout frame divided into two or more readout frame periods. FIG. 16 illustrates an embodiment in which two or more readout frame periods are divided and a corresponding photo sensor among a plurality of photo sensors is selectively sensed during each readout frame period. For example, in each of four readout frame periods Readout Frame #1 to Readout Frames #4, a corresponding photo sensor among the plurality of photo sensors may be selectively sensed.

Referring to FIG. 16, in a first readout frame period Readout Frame #1 among four readout frame periods Readout Frame #1 to Readout Frame #4, a photo sensor located in first, fifth, . . . , and (n-3)-th pixel rows (e.g., a pixel row in which the remainder is 1 when a row number is divided by 4) among a plurality of pixel rows may be sensed. In the second readout frame period Readout Frame #2, a photo sensor located in the second, sixth, . . . , and (n-2)-th pixel rows (e.g., a pixel row in which the remainder is 2 when the row number is divided by 4) among the plurality of pixel rows may be sensed. In the third readout frame period Readout Frame #3, a photo sensor located in the third, seventh, . . . , and (n-1)-th pixel rows (e.g., a pixel row in which the remainder is 3 when the row number is divided by 4) among a plurality of pixel rows may be sensed. In the fourth readout frame period Readout Frame #4, a photo sensor located in the fourth, eighth, . . . , and n-th pixel row (e.g., a pixel row in which the remainder is 0 when the row number is divided by 4) among the plurality of pixel rows may be sensed.

According to embodiments of the present disclosure, a plurality of photo sensors may be sensed in units of pixel rows in the fingerprint authentication mode. Accordingly, an image (e.g., a fingerprint image) having a relatively high sensing resolution (a first sensing resolution) may be generated.

Referring to FIG. 16, the reset frame is shown to be initiated immediately after the readout frame (e.g., the fourth readout frame Readout Frame #4), but after the readout frame, the reset frame may not be initiated and the fingerprint authentication mode may end.

In an embodiment, an area to which the first scan signals GW[p] to GW[q] of the turn-on level are applied may be limited to a partial area including an area to which a user's touch is input in the display area DA (see FIG. 4). In another embodiment, the area to which the first scan signals GW[p] to GW[q] of the turn-on level are applied may be the entire display area DA.

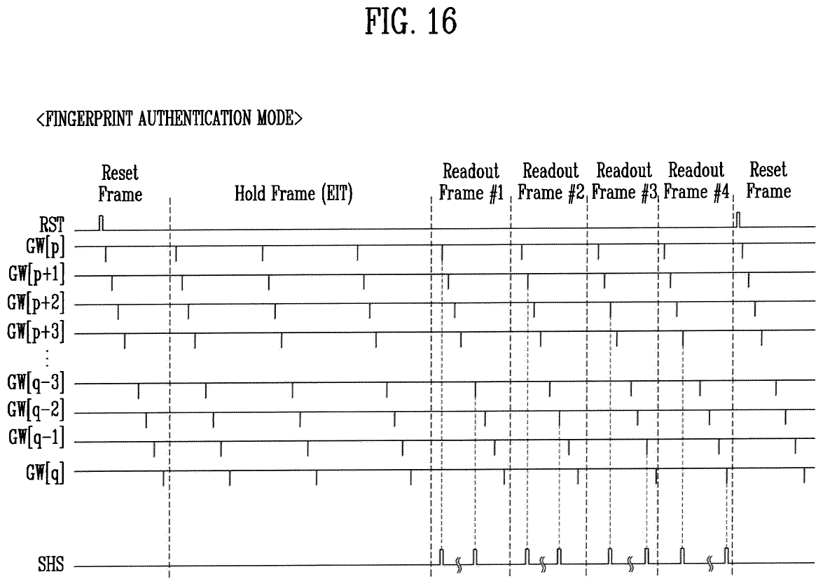

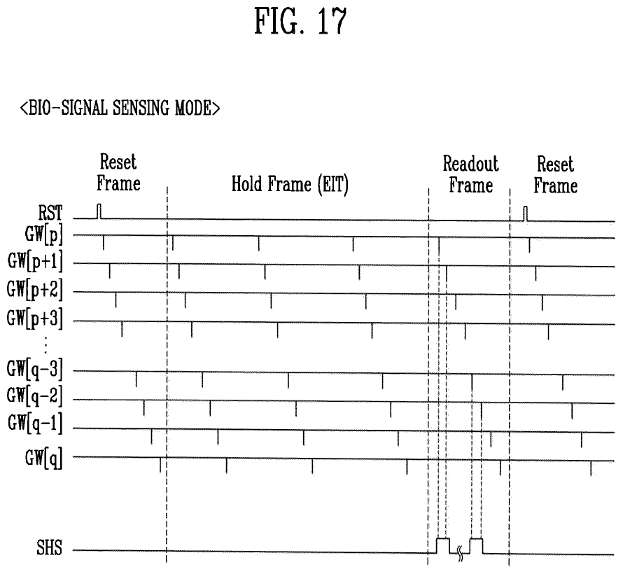

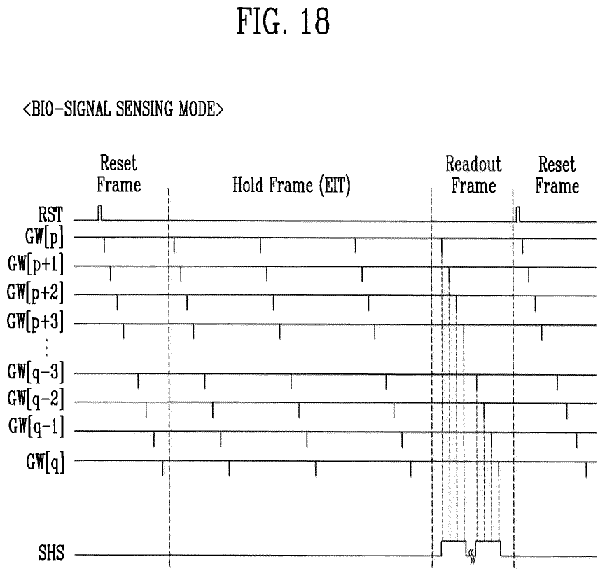

FIGS. 17 and 18 are timing diagrams of a display device for sensing a photo sensor at a second sensing resolution when a bio-signal sensing mode is selected in embodiments of the present disclosure.

The reset frame is as described above with reference to FIG. 16.

The length of the exposure time in the bio-signal sensing mode may be relatively short compared to the length of the exposure times in the fingerprint authentication mode. For example, the length of the exposure time in the bio-signal sensing mode may be about 8.3 ms (or about 1 frame based on a 120 Hz scan rate). The exposure time in the bio-signal sensing mode may include two or more hold frames.

A length of a period during which the photo sensor receives light in the bio-signal sensing mode (e.g., the period during which light is received from after the reset frame to before the readout frame) may be relatively short. The electronic device 100 (see FIG. 1) according to embodiments of the present disclosure may calculate the degree to which the amount of reflected light incident on the photo sensor changes over time in the bio-signal sensing mode. For example, the electronic device 100 may calculate the variation in the amount of reflected light incident on the photo sensor over time while operating in the bio-signal sensing mode.

The first sampling signal SHS of the turn-on level may be input to the readout circuit 450 (see FIG. 4) in a readout frame period. The first sampling signal SHS of the turn-on level may be applied for at least two horizontal periods. The horizontal period may refer to the time it takes to scan or drive one horizontal line (row) of pixels or sensors on a display panel during one frame cycle.

A description will be given with reference to FIGS. 4 and 17. The first scan signal GW[p] of the turn-on level may be applied to a p-th pixel row, and a first scan signal GW[p+1] pf the turn-on level may be apply to a (p+1)-th pixel row. In the above period, the first sampling signal SHS of the turn-on level may be input to the readout circuit 450. A first scan signal GW[q−3] of the turn-on level may be applied to the (q−3)-th pixel row, and a first scan signal GW[q−2] of the turn-on level may also be applied to a (q−2)-th pixel row. In the above period, the first sampling signal SHS of the turn-on level may be input to the readout circuit 450.

A description will be given with reference to FIGS. 4 and 18. The first scan signal GW[p] of the turn-on level may be applied to the p-th pixel row, a first scan signal GW[p+1] of the turn-on level may be apply to a (p+1)-th pixel row and a first scan signal GW[p+2] may be applied to a (p+2)-th pixel row. A first scan signal GW[p+3] may be applied to a (p+3)-th pixel row. In the above period, the first sampling signal SHS of the turn-on level may be input to the readout circuit 450. A first scan signal GW[q−3] of the turn-on level may be applied to a (q−3)-th pixel row, a first scan signal GW[q−2] of the turn-on level may be used to a (q−2)-th pixel row, a first scan signal GW[q−1] of the turn-on level may be applied to a (q−1)-th pixel row, and a first scan signal GW[q] of the turn-on level may also applied to a q-th pixel row. In the above period, the first sampling signal SHS of the turn-on level may be input to the readout circuit 450.

The electronic device 100 (see FIG. 1) according to embodiments of the present disclosure may generate an image corresponding to a bio-signal based on a result of continuously sensing two or more pixel rows in the bio-signal sensing mode. For example, the electronic device 100 may generate an image corresponding to a bio-signal by continuously sensing two or more rows of pixels in the bio-signal sensing mode and processing the resulting data. Accordingly, an image having a relatively low sensing resolution (a second sensing resolution) may be generated based on a result of integrally sensing two or more pixel rows (e.g., photo sensors located in the two or more pixel rows). For example, an image with the second sensing resolution may be generated by collectively sensing two or more pixel rows, such as photo sensors arranged in those rows.

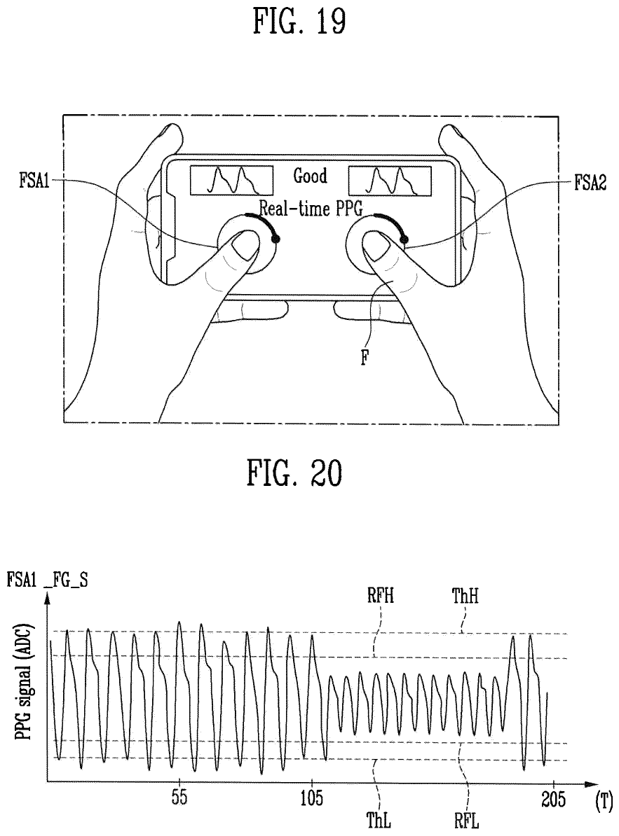

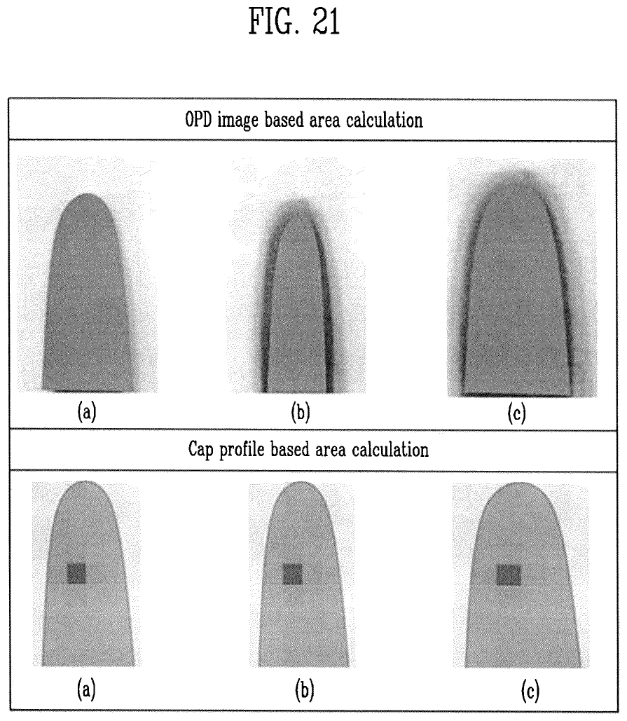

FIG. 19 is a diagram illustrating a screen displayed in a bio-signal sensing mode and a process of detecting a plurality of body part touch area. FIG. 20 is a graph illustrating one pulse signal detected in real in a biometric information measurement period and a process of detecting an inaccurate pulse signal. FIG. 21 is a diagram illustrating a detected contact area of a finger in contact with a display area during a biometric information measurement period.

Contents illustrated in FIGS. 19 to 21 are explained with reference to the preceding drawings as a whole.

During a period of sensing a bio-signal, the data driving circuit 410 may supply a data voltage Vdata to the unit pixels UP of the display panel 220. The scan driving circuit 420 and the emission driving circuit 430 may supply a scan signal and a light emission control signal to the unit pixels UP of the display panel 220, and a screen of an application for detecting the bio-signal, such as a photoplethysmogram (PPG) signal, may be displayed in the display area (DA).

The processor 130 displays a plurality of bio-signal measurement areas FSA1 and FSA2 on the application's guidance screen to guide a touch position of a body part such as a finger F. In an embodiment, the processor 130 may guide a process of detecting the photoplethysmogram signal by displaying waveforms of the detected photoplethysmogram signal (e.g., detected in real time) on the display area DA of the electronic device 100. The processor 130 may display, on a screen executing the application, the time required for bio-signal measurement, the duration of photoplethysmogram signal detection, and the first and second bio-signal measurement areas FSA1 and FSA2 using graphical elements such as circles or bar graphs.

The processor 130 may receive, via the touch driving circuit 230, a plurality of touch coordinates where a touch is detected. The processor 130 may control the display driving circuit 240 to supply a set data voltage to at least one of the sub-pixels SP1, SP2, and SP3 in a periphery of an area where the touch is detected and to detect a detection signal VCrx using at least one type of light such as green light or red light. The processor 130 may receive the detection signal VCrx from the photo sensors PHS arranged in the plurality of bio-signal measurement areas FSA1 and FSA2, respectively, via the sensing lines RX1 to RXo of the display panel 220.

The processor 130 may control the readout circuit 450 to detect photoplethysmogram signals respectively corresponding to the detection signals VCrx received corresponding to the bio-signal measurement areas FSA1 and FSA2. The processor 130 may store a digital value DSEN corresponding to the detection signal VCrx detected. The photoplethysmogram signal may correspond to a magnitude of the detection signal VCrx and its variation over time. The processor 130 may display the photoplethysmogram signals, which are detected (e.g., detected in real time), in a graphical format, such as a graph type, on the electronic device 100.

Referring to FIG. 20, the processor 130 may detect the detection signals VCrx via the photo sensor PHS for each of the bio-signal measurement areas FSA1 and FSA2 touched by the fingers F, and may perform preprocessing on the detection signals VCrx, as needed. The processor 130 may perform a set signal processing operations on each detection signal VCrx by applying preprocessing techniques such as filtering and sampling. The photoplethysmogram signals, corresponding to the magnitude and variation of the processed detection signals VCrx, may be generated, stored and monitored over preset time intervals.

The processor 130 may analyze (e.g., analyze in real time), changes in the magnitude of high and low pulses in at least one of the first and second generated and stored photoplethysmogram signals. Referring to FIG. 20, a pulse FSA1_FG_S of the first photoplethysmogram signal is shown.

The processor 130 may calculate (e.g., calculate in real time) an average magnitude value RFH of high pulses and an average magnitude value RFL of low pulses. The processor 130 may determine a normal or abnormal pulse signal detection period based on a comparison between the average magnitude value RFH of the high pulses and a predefined high threshold value ThH, and between the average magnitude value RFL of the low pulses and a predefined low threshold value ThL.

For example, the processor 130 may designate a plurality of frame periods as a normal pulse signal detection period when the average magnitude value RFH of the high pulses detected during those frame periods is equal to or greater than the predefined high threshold ThH. The processor 130 may designate a plurality of frame periods as a normal pulse signal detection period when the average magnitude value RFL of the low pulses detected during those frame periods is greater than the predefined low threshold ThL.

For example, the processor 130 may designate a plurality of frame periods as an abnormal pulse signal detection period when the average magnitude value RFH of the high pulses detected during those frame periods is smaller than the predefined high threshold ThH. The processor 130 may designate a plurality of frame periods as an abnormal pulse signal detection period when the average magnitude value RFL of the low pulses detected during those frame periods is smaller than the predefined low threshold ThL.

Referring to FIG. 21, the processor 130 may perform predefined signal processing on the detection signal VCrx detected via the photo sensor PHS at a location where a body part, such as a finger F, is detected.

In an embodiment, the processor 130 may generate an image (e.g., an image such as (a) or (c) in FIG. 21) based on the detection signal VCrx detected via the photo sensor PHS. The processor 130 may analyze a touch area or a touch shape based on a value of the detection signal VCrx detected via the photo sensor PHS and the image which is generated.

When the touch area becomes smaller than a reference area, a corresponding period may be set to the abnormal pulse signal detection period. The image generated during the corresponding period may be, for example, the image shown in (b) of FIG. 21.

In an embodiment, the processor 130 may generate a capacitance profile (Cap profile) for a signal value based on the signal value detected via the photo sensor PHS. The processor 130 may analyze the touch area or the touch shape based on the value of the detection signal VCrx detected via the photo sensor PHS and the image generated. The processor 130 may analyze the touch area or the touch shape based on the capacitance profile detected via the photo sensor PHS.

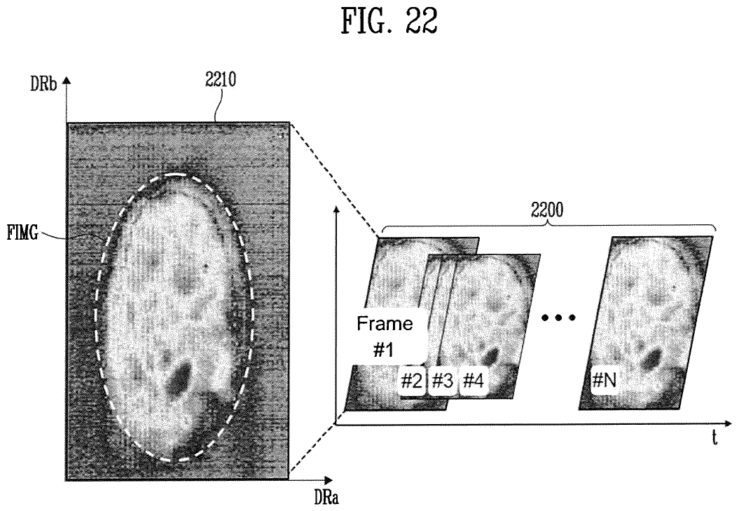

FIG. 22 is an example of images 2200 generated over a plurality of frame periods.

Referring to FIG. 22, a plurality of images 2200 are shown, and each of the plurality of images 2200 may include a finger image FIMG. For example, an image 2210 in the first frame Frame #1 may include the finger image FIMG.

The plurality of frame periods may include, for example, N (where N is an integer greater than or equal to 2) frame periods (e.g., N frame periods in sequence). The N frame periods may include the first frame Frame #1, a second frame Frame #2, a third frame Frame #3, a fourth frame Frame #4, and an N-th frame Frame #N, etc.

The finger image FIMG may be an image generated based on the detection signal VCrx (see FIG. 15) detected via the photo sensor PHS (see FIG. 4 aforementioned). the finger image FIMG may be generated by sensing light reflected from the user's finger. Shading within the finger image FIMG corresponds to a magnitude of a signal-to-noise ratio (SNR). Darker shading (or closer from yellow to red) corresponds to an area with a higher signal-to-noise ratio, while lighter shading (or closer from yellow to green or blue) corresponds to an area with a lower signal-to-noise ratio. The finger image FIMG may include an area of a low signal-to-noise ratio to an area of a high signal-to-noise ratio. For example, the processor 130 (see FIG. 1) may analyze the plurality of images 2200 on a time axis t to detect an area with the highest signal-to-noise ratio on average. For example, the processor 130 may analyze one image 2210 on a spatial axis in a first direction DRa and a second direction DRb to detect an area with the highest signal-to-noise ratio on average.

The processor 130 (see FIG. 1) may detect the area with the highest average signal-to-noise ratio and calculate changes in the magnitude of reflected light centered on the detect area. The processor 130 may interpret the finger image FIMG in a space-time axis (or a spatiotemporal domain) and, and determine the presence or absence of abnormalities in the user's finger blood vessels user's finger based on the interpreted result.

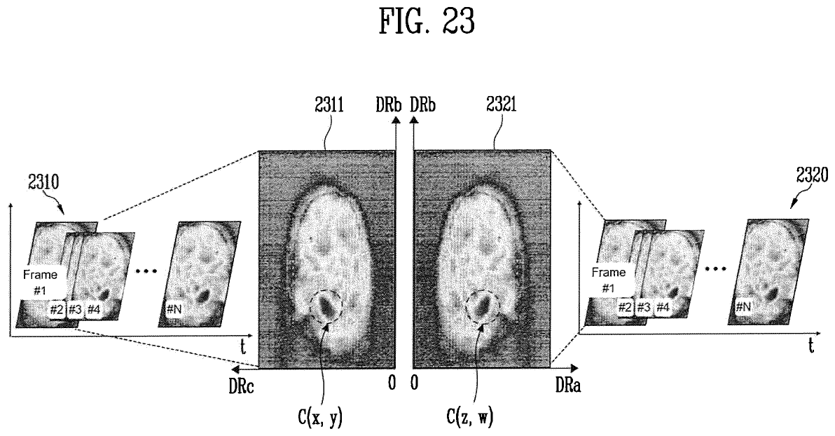

FIG. 23 is an example of a plurality of images 2310 and 2320 generated over a plurality of frame periods.

Referring to FIG. 23, a plurality of images 2310 corresponding to a left hand and a plurality of images 2320 corresponding to a right hand may be generated over N frame periods.

The plurality of images 2310 corresponding to the left hand may be displayed along a horizontal direction (e.g., a c-th direction DRc) and a vertical direction (e.g., a b-th direction DRb) as axes. The processor 130 (see FIG. 1) may detect an area with the highest signal-to-noise ratio for the plurality of images 2310 corresponding to the left hand, and calculate a center of the detected area. Referring to FIG. 23, a calculated center coordinate (e.g., a first center coordinate) of the area with the highest signal-to-noise ratio for the plurality of images 2310 corresponding to the left hand is shown to be C(x, y). For any one 2311 of the plurality of images 2310 corresponding to the left hand, an area with coordinate value (x, y) may be an area with the deepest red color.

The plurality of images 2320 corresponding to the right hand may be displayed along the horizontal direction (e.g., a a-th direction DRa) and the vertical direction (e.g., the b-th direction DRb). For example, the a-th direction DRa and the c-th direction DRc may be opposite each other, but embodiments of the present disclosure are not limited thereto. The processor 130 (see FIG. 1) may detect an area with the highest signal-to-noise ratio for the plurality of images 2320 corresponding to the right hand, and calculate a center of the detected area. Referring to FIG. 23, a calculated center coordinate (e.g., a second center coordinate) of the area with the highest signal-to-noise ratio for the plurality of images 2320 corresponding to the right hand is shown as C(z, w). For any one 2321 of the plurality of images 2320 corresponding to the right hand, an area with coordinate value (z, w) may be an area with the deepest red color.

In an embodiment, the locations of blood vessel-concentrated areas on both hands of the user may be compared by comparing the positional difference between the first center coordinate and the second center coordinate. In an embodiment, a health status of blood vessels in the user's two hands may be assessed by calculating a first feature value at and around the first center coordinate, calculating a second feature value at and around the second center coordinate, and comparing the first feature value and the second feature value with each other.

The feature value may be, for example, information related to systolic and diastolic peaks, which may be calculated using various algorithms.

In an embodiment, each of a left hand finger image and a right hand finger image is generated based on a difference in reflected light incident on the electronic device according to contraction and relaxation of blood vessels of the user.

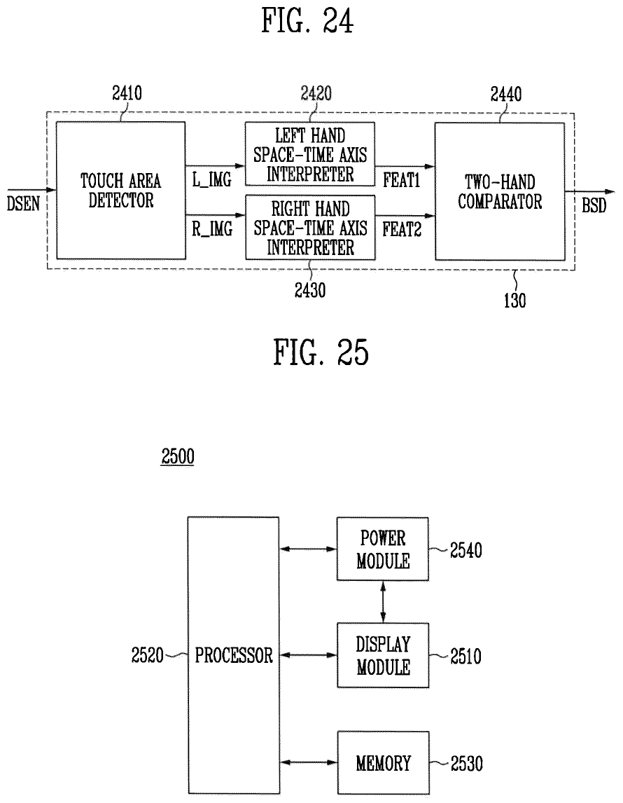

FIG. 24 is a block diagram of a processor 130 according to embodiments of the present disclosure.

Referring to FIG. 24, the processor 130 according to embodiments of the present disclosure may include a touch area detector 2410, a left hand space-time axis interpreter 2420, a right hand space-time axis interpreter 2430, and a two-hand comparator 2440. For example, the interpreters 2420 or 2430 may be implemented by a digital signal processor (ISP), a neural processing unit (NPU), an image signal processor, or an application-specific integrated circuit (ASIC). For example, the two-hand comparator 2440 may be implemented by an application processor (AP) core, an NPU, a microcontroller unit (MCU), a comparison logic circuit or a field programmable gate array (FPGA). The touch area detector 2410 may be implemented by an ISP, an AP, an NPU, an FPGA, and ASIC, or a display driver IC with an embedded microcontroller.

The touch area detector 2410 may generate left hand images L_IMG corresponding to a touch input of the left hand and right hand images R_IMG corresponding to a touch input of the right hand based on a digital value DSEN. For example, the left hand images L_IMG may include the plurality of left hand images 2310 shown in FIG. 23. For example, the right hand images R_IMG may include the plurality of right hand images 2320 shown in FIG. 23.

The left hand space-time axis interpreter 2420 may generate a first feature value FEAT1 based on the left hand images L_IMG. The first feature value FEAT1 may include information associated with a systolic peak and information associated with a diastolic peak calculated from the left hand images L_IMG. The information associated with the systolic and diastolic peaks calculated from the left hand images L_IMG may include: a magnitude of a signal at the systolic peak, a magnitude of a signal at the diastolic peak, a time taken to reach the systolic peak, a time taken to reach the diastolic peak, and an value obtained by integrating signal magnitude up to the systolic peak.

The right hand space-time axis interpreter 2430 may generate a second feature value FEAT2 based on the right hand images R_IMG. The second feature value FEAT2 may include information extracted from the systolic and diastolic peaks calculated from the right hand images R_IMG. The information associated with the systolic and diastolic peaks calculated from the right hand images R_IMG may include: a magnitude of a signal at the systolic peak, a magnitude of a signal at the diastolic peak, a time taken to reach the systolic peak, a time taken to reach the diastolic peak, and an value obtained by integrating signal magnitude up to the systolic peak.

The two-hand comparator 2440 may compare the first center coordinate and the second center coordinate to each other. The two-hand comparator 2440 may compare the first feature value FEAT1 and the second feature value FEAT2 to each other. The two-hand comparator 2440 may generate bio-signal sensing data BSD based on the first feature value FEAT1 and the second feature value FEAT2. The two-hand comparator 2440 may input the first feature value FEAT1 and the second feature value FEAT2 into a stored algorithm, such as an algorithm for calculating blood pressure. The algorithm for calculating the blood pressure may be, for example, a Deep Belief Network-Restricted Boltzmann Machine (DBN-RBM), a Convolution Neural Network (CNN), a PPG2BP-NET or a SOLED2DeepBP algorithm. However, embodiments of the present disclosure are not limited thereto, and algorithms may be variously selected by those skilled in the art within a scope of generating cardiovascular-related information (e.g., blood pressure) based on light intensity.

The two-hand comparator 2440 may calculate at least one among an Area Under the Systolic Peak (AUSP) ratio, a Rising Time (RT) difference, a Reflection Index (RI), and a Stiffness Index (SI) by using the first feature value FEAT1 and the second feature value FEAT2.

The AUSP ratio may be calculated by dividing a value, which is obtained by integrating the magnitude of the signal from the reference time point until the systolic peak of the right hand is reached, by a value, which is obtained by integrating the amplitude of the signal from the reference time point until the systolic peak of the left hand is reached.

The rising time difference may be calculated by subtracting a time which is from a reference time point until the systolic peak of the left hand is reached, from a time, which is from a reference time point until the systolic peak of the light hand is reached.

The reflection index may be calculated for each of the user's right and left hands by dividing the magnitude of the signal at the diastolic peak by the magnitude of the signal at the systolic peak.

The stiffness index may be calculated for each of the user's right and left hands by dividing the user's height by a difference between a time to reach the diastolic peak and a time to reach the systolic peak.

According to embodiments of the present disclosure, information associated with the cardiovascular system of the user's left and right hands may be generated in a space-time axis (or in a spatiotemporal domain), compared either individually, or in combination. Thereby, more diverse information related to the cardiovascular system of the user may be collected.

FIG. 25 is a block diagram of an electronic device 2500 according to an embodiment of the present disclosure.

Referring to FIG. 25, the electronic device 2500 according to an embodiment may include a display module 2510, a processor 2520, a memory 2530, and a power module 2540.

The processor 2520 may include at least one of a central processing unit (CPU), an application processor (AP), a graphics processing unit (GPU), a communication processor (CP), an image signal processor (ISP), and a controller.

The memory 2530 may store data information necessary for operation of the processor 2520 or the display module 2510. When the processor 2520 executes an application stored in the memory 2530, image data signals and/or an input control signal are transmitted to the display module 2510, and the display module 2510 may process the received signal to output image information through a display screen.

The power module 2540 may include a power supply module such as a power adapter or battery device, and a power conversion module that converts power supplied by the power supply module to generate the power required for operation of the electronic device 2500.

At least one of the above-described components of the electronic device 2500 may be included in the display device 110 (see FIG. 1) according to the above-described embodiments. In addition, some of the individual modules that are functionally included in one module may be included in the display device 110, and others may be provided separately from the display device 110. For example, display device 110 may include display module 2510, while processor 2520, memory 2530, and power module 2540 may be provided in the form of other devices in electronic device 2500 other than the display device.

In an embodiment, the processor 2520 according to embodiments of the present disclosure may correspond to the processor 130 (see FIG. 1) described above.

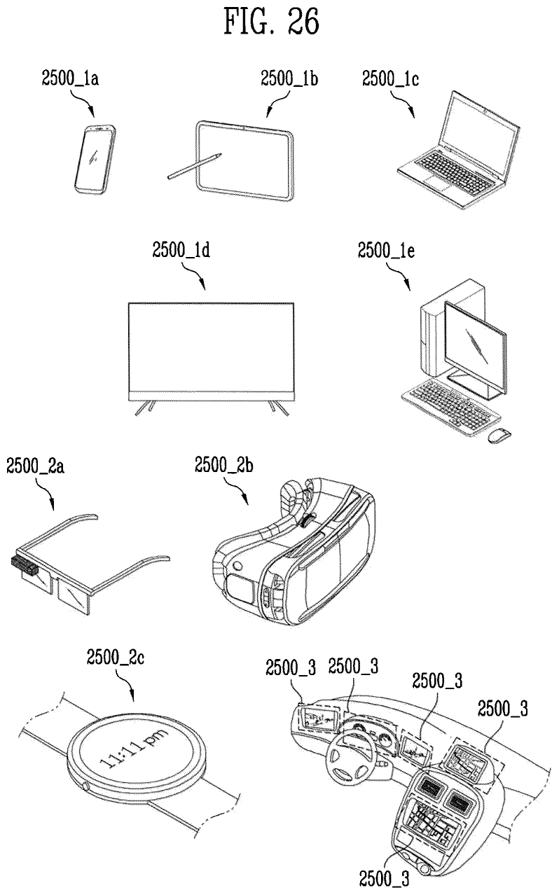

FIG. 26 is a schematic illustration of an electronic device 2500 according to various embodiments.

Referring to FIG. 26, various electronic devices to which a display device according to embodiments is applied may include not only an electronic device for displaying an image such as a smartphone 2500_1a, a tablet PC 2500_1b, a laptop 2500_1c, a TV 2500_1d, and a desk monitor 2500_1e, but also a wearable electronic device including a display module such as smart glasses 2500_2a, a head mounted display 2500_2b, and a smart watch 2500_2c, a vehicle electronic device 2500_3 including a display module, such as a CID (Center Information Display) disposed on an instrument panel, a center fascia, and a dashboard of a vehicle, a room mirror display, and the like.

According to embodiments of the present disclosure, the electronic device and the driving method thereof may compare information acquired from the left side of the user's body and information acquired from the right side of the user's body over a specific period of time in terms of time and space.

The drawings and detailed description of the invention referred to so far are merely illustrative of the invention, which has been used merely for the purpose of describing the invention and not for the purpose of limiting the scope of the invention. Therefore, those skilled in the art will appreciate that various modifications and equivalent embodiments are possible therefrom.

What is claimed is:

1. An electronic device comprising:
a display panel having a plurality of sub-pixels and a plurality of photo sensors;
a display driving circuit configured to drive the plurality of sub-pixels and the plurality of photo sensors;
a touch panel positioned to overlap the display panel and having a plurality of touch electrodes;
a touch driving circuit to drive the plurality of touch electrodes to respectively detect an area where a finger of a user's left hand has been touched and an area where a finger of the user's right hand has been touched; and
a processor controlling the display driving circuit over a plurality of frame periods to generate a left hand finger image in an area where the finger of the user's left hand is detected, generate a right hand finger image in an area where the finger of the user's right hand is detected, and compare the generated left and right hand finger images.

2. The electronic device of claim 1, wherein the processor generates a plurality of images including the left hand finger image, and a plurality of images including the right hand finger image during the plurality of frame periods.

3. The electronic device of claim 2, wherein the processor is configured to:
detect an area having a highest signal-to-noise ratio in the plurality of images including the left hand finger image, and
detect an area having a highest signal-to-noise ratio in the plurality of images including the right hand finger image.

4. The electronic device of claim 3, wherein the processor is configured to:
calculate a first feature value around an area having the highest signal-to-noise ratio in the left hand finger image, and
calculate a second feature value around an area having the highest signal-to-noise ratio in the right hand finger image.

5. The electronic device of claim 4, wherein the first feature value includes information associated with a systolic peak and information associated with a diastolic peak which are measured from the user's left hand, and
wherein the second feature value includes information associated with a systolic peak and information associated with a diastolic peak which are measured from the user's right hand.

6. The electronic device of claim 5, wherein the processor generates bio-signal sensing data based on the first feature value and the second feature value.

7. The electronic device of claim 6, wherein the bio-signal sensing data includes information associated with a cardiovascular system of the user.

8. The electronic device of claim 1, wherein the processor comprises:
a touch area detector to receive a digital value from the display driving circuit, to generate left hand images including the left hand finger image in an area where the finger of the user's left hand is detected, and to generate right hand images including the right hand finger image in an area where the finger of the user's right hand is detected;
a left hand space-time axis interpreter configured to generate a first feature value from the left hand images generated over a plurality of frame periods in spatial coordinates;
a right hand space-time axis interpreter configured to generate a second feature value from the right hand images generated over a plurality of frame periods in spatial coordinates; and
a two-hand comparator configured to generate bio-signal sensing data based on the first feature value and the second feature value.

9. The electronic device of claim 8, wherein the left hand space-time axis interpreter detects an area having a highest signal-to-noise ratio in the left hand images, and
wherein the right hand space-time axis interpreter detects an area having a highest signal-to-noise ratio in the right hand images.

10. The electronic device of claim 9, wherein the left hand space-time axis interpreter generates the first feature value centered on a first center coordinate calculated to have the highest signal-to-noise ratio in the left hand images, and
wherein the right hand space-time axis interpreter generates the first feature value centered on a second center coordinate calculated to have the highest signal-to-noise ratio in the right hand images.

11. The electronic device of claim 10, wherein the two-hand comparator compares positions of the first center coordinate and the second center coordinate.

12. The electronic device of claim 9, wherein the two-hand comparator compares the first feature value with the second feature value.

13. A driving method of an electronic device including a memory storing an application and a processor executing the application, the driving method comprising:
generating left hand images including a left hand finger image of a user in an area where a finger of the user's left hand is detected to have been touched;
generating right hand images including a right hand finger image of the user in an area where a finger of the user's right hand is detected to have been touched;
generating a first feature value from the left hand images generated over a plurality of frame periods in spatial coordinates;
generating a second feature value from the right hand images generated over a plurality of frame periods in spatial coordinates; and
generating bio-signal sensing data based on the first feature value and the second feature value.

14. The driving method of claim 13, wherein, in the generating a first feature value from the left hand images, the processor detects an area having a highest signal-to-noise ratio in the left hand images, and
wherein, in the generating a second feature value from the right hand images, the processor detects an area having a highest signal-to-noise ratio in the right hand images.

15. The driving method of claim 14, wherein, in the generating a first feature value from the left hand images, the processor generates the first feature value centered on a first center coordinate calculated to have the highest signal-to-noise ratio in the left hand images, and
wherein, in the generating a second feature value from the right hand images, the processor generates the second feature value centered on a second center coordinate calculated to have the highest signal-to-noise ratio in the right hand images.

16. The driving method of claim 15, wherein, in the generating bio-signal sensing data, the processor compares positions of the first center coordinate and the second center coordinate.

17. The driving method of claim 14, wherein, in the generating bio-signal sensing data, the processor compares the first feature value and the second feature value.

18. The driving method of claim 13, wherein the first feature value includes information associated with a systolic peak and information associated with a diastolic peak which are measured from the user's left hand, and
wherein the second feature value includes information associated with a systolic peak and information associated with a diastolic peak which are measured from the user's right hand.

19. The driving method of claim 13, wherein the bio-signal sensing data includes information associated with a cardiovascular system of the user.

20. The driving method of claim 13, wherein each of the left hand finger image and the right hand finger image is generated based on a difference in reflected light incident on the electronic device according to contraction and relaxation of blood vessels of the user.
